(19) United States
(12) Patent Application Publication
(10) Pub. No.: US 2018/0340163 A1
(43) Pub. Date: Nov. 29, 2018

(54) RECOMBINANT SERINE PROTEASES

(71) Applicant: ACADEMISCH ZIEKENHUIS LEIDEN, Leiden (NL)

(72) Inventors: Daniel VERHOEF, Leiden (NL); Pieter H REITSMA, Leiden (NL); Mettine H.A. BOS, Leiden (NL)

(21) Appl. No.: 15/777,963

(22) PCT Filed: Nov. 25, 2016
(86) PCT No.: PCT/NL2016/050833

§ 371 Date: May 22, 2018

(30) Foreign Application Priority Data
Nov. 25, 2015 (EP) 15196291.7

(51) Int. Cl. C12N 9/74; C12N 9/64; C12N 9/76; C12N 9/72

(52) U.S. Cl. CPC C12N 9/6429; C12N 9/6443; C12N 9/6427; C12N 9/6462; A61K 38/00; C12Y 304/21005; C12Y 304/21027; C12Y 304/21073; C12Y 304/21004

(57) ABSTRACT

The present invention relates to recombinant proteins having serine protease polypeptides that have serine protease activity in the presence of a serine protease inhibitor and that are able to completely or partially reverse a serine protease inhibitor effect, for example in a subject treated with a serine protease inhibitor. More specifically, described herein are recombinant proteins and methods for completely or partially reversing an anti-coagulant effect of a coagulation inhibitor.

### FIELD OF THE INVENTION

The present invention is in the field of preparations for medical purposes. More specifically, the invention relates to specific recombinant serine proteases that have serine protease activity in the presence of serine protease inhibitors.

### STATE OF THE ART

Currently, a steadily increasing number of serine protease inhibitors are being developed that prevent or inhibit serine proteases from performing their protease activity. Serine proteases (or serine endopeptidases) are enzymes that cleave peptide bonds in proteins, in which serine serves as the nucleophilic amino acid at the protease's active site (Hedstrom, 2002. Chem Rev 102: 4501-4524). In humans, serine proteases are responsible for coordinating various physiological processes, including digestion, immune response, blood coagulation and reproduction (Hedstrom, 2002. Chem Rev 102: 4501-4524). Some well-known serine proteases include thrombin, blood coagulation factor XIa, urokinase-type plasminogen activator and trypsin, the first two being involved in blood coagulation.

Serine proteases can be inhibited by a diverse group of inhibitors, including synthetic chemical inhibitors and natural proteinaceous inhibitors. One family of natural inhibitors called “serpins” (abbreviated from serine protease inhibitors) can form a covalent bond with a serine protease, thereby inhibiting its function. Some of the best-studied serine protease inhibitors are antithrombin and alpha 1-antitrypsin, known for their role in blood coagulation and emphysema, respectively.

Direct serine protease inhibitors, such as direct thrombin inhibitors (DTI), are being developed and are expected to largely replace the classic oral anticoagulants such as antithrombin in the near future, because of their rapid therapeutic effectiveness, ease of dosing, and lack of monitoring requirements (He et al., 2015. Molecules 20, 11046-11062; Wang et al., 2015. Arch Pharm 348: 595-605). These inhibitors target the active site of the serine protease and are generally small molecules that are suitable for oral administration. Univalent DTIs include, among others, Argatroban, Melagatran—or its prodrug Ximelagatran —, Dabigatran—or its prodrug Dabigatran Etexilate—or analogs thereof, peptide or peptidomimetic inhibitors (Mehta et al., 2014. Expert Opin Ther Pat 24: 47-67), RWJ-671818 or analogs thereof (Lu et al., 2010. J Med Chem 53: 1843-1856), 3-(2-Phenethylamino)-6-methyl-1-(2-amino-6-methyl-5-methylenecarboxamidomethylpyridinyl)pyrazinone or analogs thereof, (Sanderson et al., 1998. J Med Chem 41: 4466-4474), (E)-N-(3-((1-(benzo[b]thiophen-2-ylmethyl)-1H-1,2,3-triazol-4-yl)methoxy)phenyl)-2-(3-chlorophenyl)ethenesulfonamide or analogs thereof (Siles et al., 2011. Bioorg med Chem Lett 21: 5305-5309), N-{3-[(3-fluorobenzyl)oxy]phenyl}-1-pyridin-4-ylpiperidine-4-carboxamide or analogs thereof (de Candia et al., 2013. J Med Chem 56: 8696-8711), and compound 2 by Merck or analogs thereof (Morrissette et al., 2004. Bioorg Med Chem Lett 14: 4161-4164). Univalent DTIs have specifically been designed to tightly bind to the active site of thrombin and halt its protease activity.

Univalent direct inhibitors have also been found for coagulation factor XIa and include, among others, 4, 5, 6-trisubstituted pyrimidine derivatives (U.S. Pat. No. 8,609,676 B2), BMS-262084 (Schumacher et al., 2007. Eur J Pharmacol 570: 167-174), compound 1 of Bristol-Meyers Squibb or analogs thereof (Quan et al., J Med Chem 2014. 57: 955-969), compounds 2 and 33 of Bristol-Meyers Squibb or analogs thereof (Pinto et al., 2015. Bioorg Med Chem Lett 25: 1635-1642), compound 13 of AstraZeneca or analogs thereof (Fjellstrom et al., 2015. PLoS One 10: e0113705), aryl boronic acids (Lazarova et al., 2006. Bioorg Med Chem Lett 16: 5022-5027), and macrocyclic indoles (Hanessian et al., 2010. Bioorg Med Chem Lett 20: 6925-6928).

Univalent inhibitors for urokinase-type plasminogen activator include, among others, WX-UK1—or its prodrug Upamostat, also known as Mesupron or WX-671—, and APC-10302 or analogs thereof (Katz et al., 2001. Chem Biol 8: 1107-1121). Interestingly, it was found that the direct thrombin inhibitor Melagatran and the direct urokinase-type plasminogen activator inhibitor APC-10302 are also capable of binding to the active site of trypsin and inhibiting its serine protease activity (Dullweber et al. 2001. J Mol Biol 313: 593-614; Katz et al., 2001. Chem Biol 8: 1107-1121.

A drawback to the use of serine protease inhibitors is that effective restoration of normal serine protease activity generally requires either full replacement of serine protease, or effective removal of inhibitor from the subject. This is a disadvantage, as the induction of serine protease activity after inhibition should preferably be instantly and directly achievable, instead of gradually over time, both in a clinical and non-clinical setting. For example, in the context of anticoagulant therapy, a general lack of specific reversal strategies may result in potential life-threatening bleeding complications after application of an inhibitor of, for example, thrombin. The latter is exemplified by the fact that, alone in the Netherlands, annually over 5,000 patients treated with anticoagulants suffer from an severe, adverse bleeding event, including over 800 fatalities (Adriaansen H., et al.: “Samenvatting Medische Jaarverslagen van de Federatie van Nederlandse Trombosediensten”, 2014; 1-38).

At this moment, a direct and instant reversal strategy to prevent and halt the inhibitory effect of serine protease inhibitors is not available.

The present invention solves this problem by providing, as an adequate reversal strategy to prevent and halt the inhibitory effect of serine protease inhibitors, a recombinant protein comprising a serine protease polypeptide selected from the group comprising thrombin, coagulation factor XIa, urokinase-type plasminogen activator and trypsin, said polypeptide comprising an insertion of at least one amino acid residue in an outer-surface peptide structure, wherein said peptide structure is a region of amino acid residues between Gly-427 and Asp-462, preferably between His-450 and Asp-462, more preferably between His-450 and Leu-459, of SEQ ID NO: 1; a region of amino acid residues between Val-463 and Asp-480, preferably between His-469 and Asp-480 or Ser-477, of SEQ ID NO: 2; a region of amino acid residues between Leu-73 and Asp-107, preferably between His-76 and Asp-107, more preferably between Gln-87 and Asp-107, and most preferably between His-96 and Leu-104 or Asp-107, of SEQ ID NO: 3; a region of amino acid residues between Val-237 and Asp-275, preferably between Phe-254 or Val-256 and Asp-275 or Asn-274, more preferably between His-262 and Asp-275, Asn-274 or Ala-271, of SEQ ID NO:4.

Preferably, the serine protease polypeptide is selected from the group consisting of thrombin, coagulation factor XIa, trypsin and urokinase-type plasminogen activator, preferably human thrombin, human coagulation factor XIa, human trypsin and human urokinase-type plasminogen activator. It is noted that SEQ ID NO: 1 provides the amino acid sequence of human prothrombin, SEQ ID NO: 2 provides the amino acid sequence of human coagulation factor XI, SEQ ID NO: 3 provides the amino acid sequence of human trypsin-1 and SEQ ID NO:4 provides the amino acid sequence of human urokinase-type plasminogen activator.

It was found that a serine protease polypeptide selected from the group formed by thrombin, coagulation factor XIa, trypsin and urokinase-type plasminogen activator, with an altered amino acid composition, preferably an insertion of at least one amino acid, in a region between Gly-427 and Asp-462 of SEQ ID NO: 1 for thrombin, in a region between Val-463 and Asp-480 or Ser-477 of SEQ ID NO: 2 for coagulation factor XIa, in a region between Leu-73 and Asp-107 of SEQ ID NO: 3 for trypsin or in a region between Val-237 and Asp-275 of SEQ ID NO:4 for urokinase-type plasminogen activator, are catalytically active in the presence of a serine protease inhibitor. This is surprising because the available crystal structures of a serine protease in complex with its inhibitor indicate that the amino acid residues in the outer-surface peptide structure do not contact the inhibitor (see, for examples, FIGS. 2-4).

In addition, the amino acid residues in the outer-surface peptide structure seem to form a flexible loop structure of which neither the composition, i.e. identity of amino acid residues, nor the number of amino acid residues, is conserved between thrombin, human coagulation factor XIa, human trypsin and human urokinase-type plasminogen activator. The alteration of this loop, by insertion of at least one amino acid residue, was not thought to alter the binding of an inhibitor to the serine protease and/or the activity of the serine protease after binding of an inhibitor.

In addition, known inhibitors of thrombin, human coagulation factor XIa, human trypsin and urokinase-type plasminogen activator are structurally unrelated compounds. This further renders it unlikely, a priori, that insertion of at least one amino acid residue in an outer-surface peptide structure of a thrombin, coagulation factor XIa trypsin or urokinase-type plasminogen activator serine protease results in a serine protease with decreased sensitivity to inhibition by the inhibitor.

The recombinant serine protease has a decreased sensitivity to inhibition by serine protease inhibitors compared to a serine protease not having said altered amino acid composition. The present invention provides therefore an antidote to a serine protease inhibitor, that does not depend on the generation of free, endogenous serine protease and offers a fast and direct reversal strategy to prevent and stop the inhibitory effect of serine protease inhibitors.

The term “serine protease”, as used herein, refers to an enzyme that degrades proteins by hydrolyzing peptide bonds and is primarily characterized by having an active serine residue in the active site. Serine proteases are also commonly referred to as a serine endopeptidases. Preferably, the term “serine protease” refers to enzymes having a spatial arrangement of the catalytic triad amino acid residues histidine, aspartic acid and serine, which are, by way of example, indicated as His-406, Asp-462 and Ser-568 in SEQ ID NO:1. The skilled person can easily assess and find catalytic triad amino acid residues in serine endopeptidases The term includes serine endopeptidases comprised in the enzyme class (EC) 3.4.21, such as chymotrypsin, trypsin, thrombin, coagulation factor VIIa, coagulation factor IXa, coagulation factor XIa, elastase, granzyme A, granzyme B, kallikrein 8 and precursors of these serine proteases such as inactive prepro- and pro-precursors. Preferably, the serine protease polypeptide is a thrombin, coagulation factor XIa, trypsin or urokinase-type plasminogen activator polypeptide, preferably selected from the group consisting of thrombin, coagulation factor XIa, trypsin or urokinase-type plasminogen activator polypeptides. Methods to determine whether a protein is a serine protease are known in the art and include sequence comparison and use of a protease detection kit, for example of Sigma-Aldrich.

The amino acid sequence of human prothrombin is provided in SEQ ID NO: 1 and can be found in GENBANK® under accession number AAC63054.1. Prothrombin with the sequence listed in SEQ ID NO: 1 is a precursor containing a prepro-leader sequence (amino acid residues 1 to 43 of SEQ ID NO: 1), followed by sequences corresponding to an activation peptide fragment 1 (amino acid residues 44-198) and a subsequent activation peptide fragment 2 (amino acid residues 199-327), followed by a thrombin light chain (amino acid residues 328-363) and a thrombin heavy chain (amino acid residues 364-622). The term “prothrombin”, as used herein, refers to an inactive prothrombin precursor protein. The term “thrombin”, as used herein refers to the catalytically active form of a prothrombin having a thrombin light and heavy chain. According to the definitions used herein, a prothrombin comprises a thrombin polypeptide.

In the context of the invention, a protein is a prothrombin or thrombin polypeptide if it is a procoagulant serine protease, preferably which may cleave Arg-|-Gly bonds in fibrinogen to form fibrin and releases fibrinopeptides A and B. A thrombin, also termed fibrinogenase, preferably comprises stretches of amino acid residues that correspond to stretches of amino acid residues that are conserved between prothrombin or thrombin polypeptides of different species. For example, a procoagulant serine protease comprising a polypeptide that contains stretches of amino acid residues that correspond to amino acid residues Arg-338 to Glu-343, Pro-376 to Leu-381, Ser-385 to Leu-395, Arg-461 to Leu-465, Lys-498 to Leu-507, Lys-559 to Lys-575 and Gly-586 to Arg-596 of SEQ ID NO:1 is assumed to be a prothrombin or thrombin polypeptide. The term “prothrombin” or “thrombin” includes reference to serine proteases referred to in EC 3.4.21.5.

The skilled person can corroborate whether a serine protease is indeed a thrombin, for example by testing proteolytic cleavage on a substrate suitable for that purpose such as, for example, the (i) S-2238™ substrate of Chromogenix (brand of Instrumentation Laboratory (Bedford, Mass., USA) with the formula Bz-IIe-Glu(gamma-OR)-Gly-Arg-pNA .HCl (R═H (50%) and R═CH3 (50%); molecular weight: 741.3; part Number: 82 0316 39), for thrombin while following the manufacturer's instructions, and/or (ii) Pefachrome® TH series chromogenic substrates (DSM Nutritional Products, CH), with chemical formulas Tos-Gly-Pro-Arg-pNA AcOH; H-D-CHG-Ala-Arg-pNA .2AcOH, H-D-CHG-But-Arg-pNA 2AcOH; H-D-CHG-Pro-Arg-pNA .2AcOH, H-D-CHA-Ala-Arg-pNA 2AcOH; H-D-CHA-Gly-Arg-pNA .2AcOH; CH₃OCO-Gly-Pro-Arg-pNA AcOH and/or H-beta-Ala-Gly-Arg-pNA .2AcOH.

The amino acid sequence of human coagulation factor XI is provided in SEQ ID NO: 2 and can be found in GENBANK® under accession number AAA51985.1. Coagulation factor XI having the sequence provided in SEQ ID NO: 2 is a precursor protein containing a signal peptide (amino acid residues 1-18), a coagulation factor XIa heavy chain (amino acid residues 19-387) and a coagulation factor XIa light chain (amino acid residues 388-625). The term “coagulation factor XI” as used herein, refers to an inactive coagulation factor XI precursor protein. The term “coagulation factor XIa”, as used herein refers to the catalytically active form of a coagulation factor XI having a coagulation factor XIa heavy chain and a coagulation factor XIa light chain. According to the definitions used herein, a coagulation factor XI comprises a coagulation factor XIa polypeptide.

In the context of the invention, a protein is considered to be a coagulation factor XI or coagulation factor XIa polypeptide if it is procoagulant that selectively cleaves Arg-|-Ala and Arg-|-Val bonds in factor IX to form factor IXa. The full-length amino acid sequence of said protein preferably comprises stretches of amino acid residues that correspond to stretches of amino acid residues that are conserved between coagulation factor XI or coagulation factor XIa polypeptides of different species. For example, a procoagulant serine protease comprising a polypeptide that contains a stretch of amino acid residues that corresponds to amino acid residues Asp-480 to Ala-482, Cys-560 to Gly-562, and Gly-573 to Leu-579 of SEQ ID NO:2 is assumed to be a coagulation factor XI or coagulation factor XIa polypeptide. The term “coagulation factor XI” or “coagulation factor XIa” includes reference to serine proteases referred to in EC 3.4.21.27. The skilled person can corroborate whether a serine protease is a coagulation factor XI or coagulation factor XIa polypeptide, for example by testing proteolytic cleavage on a substrate suitable for that purpose such as, for example, (i) the S2366™ substrate of Chromogenix (brand of Instrumentation Laboratory (Bedford, Mass., USA) with the formula pyroGlu-Pro-Arg-pNA.HCl (molecular weight of 539.0; Part Number 82 1090 39), while following the manufacturer's instructions, and/or (ii) the Pefachrome® FXIa chromogenic substrate (DSM Nutritional Products, CH), with chemical formula Z-Aad-Pro-Arg-pNA AcOH).

The amino acid sequence of human trypsin-1 is provided in SEQ ID NO: 3 and can be found in GENBANK® under “NP_002760.1”. Trypsin-1 with the sequence provided in SEQ ID NO:3 is a precursor protein having a signal peptide (amino acid residues 1-15), an activation peptide (amino acid residues 16-23), an alpha-trypsin chain 1 (amino acid residues 24-122) and an alpha-trypsin chain 2 (amino acid residues 123-247). The two-chain form is produced by proteolytic cleavage after residue Arg-122 of SEQ ID NO: 3. It is noted that the alpha-trypsin chains can exist as one catalytically active peptide chain (amino acid residues 24-247). It is generally known that trypsin is the archetype of serine proteases. The term “trypsin”, as used herein, refers to the inactive trypsin precursor protein, the catalytically active single chain form and the catalytically active two-chain form having a separate alpha-trypsin chain 1 and an alpha-trypsin chain 2.

In the context of the invention, a protein is considered to be a trypsin polypeptide if it is a serine protease that preferentially cleaves Arg-|-Xaa, Lys-|-Xaa. The full-length amino acid sequence of said protein preferably comprises stretches of amino acid residues that correspond to stretches of amino acid residues that are conserved between trypsin polypeptides of different species. For example, a serine protease comprising a polypeptide that contains stretches of amino acid residues that correspond to amino acid residues Phe-47 to Gly-50, Gly-191 to Gln-197, Asp-199 to Pro-203, and Val-214-Gly-217 of SEQ ID NO:3 is assumed to be a trypsin polypeptide. The term “trypsin” includes reference to serine proteases that preferentially cleave Arg-|-Xaa, Lys-|-Xaa and/or are listed in EC 3.4.21.4. The term “trypsin” thus includes reference to trypsin proteins other than trypsin 1, such as trypsin 2, trypsin 3, trypsin 3, trypsin 4, trypsin 5 and trypsin 6. Said trypsin preferably is trypsin 1. The skilled person can corroborate whether a serine protease is a trypsin, for example by testing proteolytic cleavage on a substrate suitable for that purpose such as, for example, (i) the S-2222™ substrate of Chromogenix (brand of Instrumentation Laboratory (Bedford, Mass., USA) with the formula: Bz-Ile-Glu(gamma-OR)-Gly-Arg-pNA.HCl (R═H (50%) and R═CH₃ (50%) with a molecular weight of 741.3; catalog number S820316), while following the manufacturer's instructions, and/or the Pefachrome® TRY (trypsin) chromogenic substrate (DSM Nutritional Products, CH), with chemical formula Cbo-Val-Gly-Arg-pNA AcOH.

The amino acid sequence of human urokinase-type plasminogen activator is provided in SEQ ID NO:4 and can be found in GENBANK® under “AAK53822.1”. Urokinase-type plasminogen activator with the sequence provided in SEQ ID NO:4 is a precursor protein having a signal peptide (amino acid residues 1-20), and a chain (amino acid residues 21-431) which can be subdivided in a long chain A (amino acid residues 21-177), a short chain A (amino acid residues 156-177) and a chain B (amino acid residues 179-431). The term “urokinase-type plasminogen activator”, as used herein, refers to the inactive urokinase-type plasminogen activator precursor protein and its catalytically active chain form.

In the context of the invention, a protein is considered to be a urokinase-type plasminogen activator if it is a serine protease that specifically cleaves Arg-|-Val bonds in plasminogen to form plasmin. The full-length amino acid sequence of said protein preferably comprises stretches of amino acid residues that correspond to stretches of amino acid residues that are conserved between urokinase-type plasminogen activator polypeptides of different species. For example, a serine protease comprising a polypeptide that contains stretches of amino acid residues that correspond to amino acid residues His-119 to Asn-124 and/or Asn-274 to Leu-278 of SEQ ID NO:4 and which specifically cleaves Arg-1-Val bonds in plasminogen to form plasmin, is assumed to be a urokinase-type plasminogen activator polypeptide.

The skilled person can corroborate whether a serine protease is a urokinase-type plasminogen activator, for example by testing proteolytic cleavage on a substrate suitable for that purpose such as, for example, (i) the S2444™ substrate of Chromogenix (brand of Instrumentation Laboratory (Bedford, Mass., USA) with the formula Glu-Gly-Arg-pNA HCl (molecular weight of 498.9), while following the manufacturer's instructions, and/or (ii) the Pefachrome® uPA—series chromogenic substrates for urokinase-type plasminogen activator (DSM Nutritional Products, CH), with chemical formula Bz-beta-Ala-Gly-Arg-pNA. AcOH and/or Cbo-Glu(OtBu)-Gly-Arg-pNA AcOH.

The term “recombinant”, as used herein, refers to a protein that is produced using recombinant DNA techniques known to the person skilled in the art. A recombinant protein preferably is not identical to a native protein, for example because the amino acid composition differs due to exchanges of amino acid residues and/or deletion or insertion of one or more amino acid residues, and/or because of a difference in posttranslational modification such as glycosylation.

The phrase “recombinant protein comprising a serine protease”, as used herein, is meant to encompass a protein that comprises a recombinant serine protease polypeptide, preferably of mammalian, more preferably primate, and most preferably of human origin. The phrase includes, for example, a recombinant mammalian serine protease precursor protein, such as prothrombin, that is processed and/or activated into a mammalian thrombin polypeptide. Thus, a protein of the invention preferably is a recombinant mammalian, preferably primate, more preferably human or humanized, thrombin, coagulation factor XIa, trypsin or urokinase-type plasminogen activator comprising an insertion of at least one amino acid residue in an outer-surface peptide structure, wherein said peptide structure is a region of amino acid residues corresponding to the region of amino acid residues between Gly-427 and Asp-462, preferably between His-450 and Asp-462, more preferably between His-450 and Leu-459, of SEQ ID NO: 1 for thrombin; a region of amino acid residues corresponding to the region of amino acid residues between Val-463 and Asp-480, preferably between His-469 and Asp-480 or Ser-477, of SEQ ID NO: 2 for coagulation factor XIa; a region of amino acid residues corresponding to the region of amino acid residues between Leu-73 and Asp-107, preferably between His-96 and Asp-107 or Leu-104, of SEQ ID NO: 3 for trypsin; and a region of amino acid residues corresponding to the region between Val-237 and Asp-275, preferably between Phe-254 or Val-256 and Asp-275 or Asn-274, more preferably between His-262 and Asp-275 or Asn-274, of SEQ ID NO:4 of urokinase-type plasminogen activator. In addition, said phrase includes a protein that comprises one or more additional amino acid sequences, besides the serine protease polypeptide, for example an amino acid sequence that constitutes a tag, for example a FLAG tag as described in EP0150126, and/or one or more other identification peptides.

The term “humanized”, as is used herein, refers to the replacement or humanization of preferably exterior amino acid residues of a protein of one species for amino acid residues that are present in a human homologue of the protein so that the protein of the first species will not be immunogenic, or is less immunogenic, when applied to a human. The replacement of exterior residues preferably has little, or no, effect on the interior domains, or on the interdomain contacts between light and heavy chains A protein of the invention of non-human origin, preferably mammalian origin, more preferably primate origin, is preferably humanized in order to reduce the immunogenicity of said protein when applied to a human.

A non-human protein of the invention preferably comprises a humanized mammalian, more preferably a humanized primate, serine protease polypeptide, as the risk of an antigenic response upon administration in the human body is expected to be lower as compared to a protein of the invention comprising a non-humanized serine protease polypeptide.

In the context of humanizing proteins, attention can be paid to the process of humanizing that is applicable to antibodies. This process makes use of the available sequence data for human antibody variable domains compiled by Kabat et al. (1987) Sequences of Proteins of Immunological Interest, 4th ed., Bethesda, Md., National Institutes of Health, updates to this database, and other accessible U.S. and foreign databases (both nucleic acid and protein). Non-limiting examples of the methods used to generate humanized antibodies include EP 519596; U.S. Pat. No. 6,797,492; and Padlan et al., 1991. Mol Immunol 28: 489-498. Further exemplifying the process of humanization of non-human proteins, Sarkar et al., 2012, Journal of Lipids, Article ID 610937, p. 1-13 described that Paraoxonase-1 was successfully humanized by altering the surface of the enzyme to reflect the human sequence.

The term “serine protease inhibitor”, as used herein, refers to an agent that is capable of inhibiting serine protease activity. Preferably, the serine protease inhibitor is an univalent direct serine protease inhibitor, preferably a small molecule or a peptide or peptidomimetic, which acts by binding to the active site of a serine protease. Preferably, the serine protease inhibitor is a thrombin inhibitor, coagulation factor XIa inhibitor, trypsin inhibitor or urokinase-type plasminogen activator inhibitor.

The term “thrombin inhibitor”, as used herein, includes, but is not limited to direct thrombin inhibitors, which include bivalent direct thrombin inhibitors such as hirudin, bivalirudin, and lepirudin, which act by binding to the active site of thrombin and by binding to exosite 1 of thrombin. Exosite 1 is functionally inaccessible in prothrombin and becomes exposed upon activation (Huntington, 2005. J Thromb Haemos 3: 1861-1872; Lane et al., 2005. Blood: 106: 2605-2612). The term “thrombin inhibitor” further includes reference to, and preferably is, an univalent direct thrombin inhibitor which acts by binding to the active site of thrombin. These univalent direct thrombin inhibitors include argatroban ((2R,4R)-1-[(2S)-5-(diaminomethylideneamino)-2-[(3-methyl-1,2,3,4-tetrahydroquinolin-8-yl)sulfonylamino]pentanoyl]-4-methyl-piperidine-2-carboxylic acid) or biologically active analogs thereof; melagatran (2-[[(1R)-2-[(2S)-2-[(4-carbamimidoylphenyl)methylcarbamoyl]azetidin-1-yl]-1-cyclohexyl-2-oxoethyl]amino]acetic acid) and its prodrug ximelagatran (ethyl 2-[[(1R)-1-cyclohexyl-2-[(2S)-2-[[4-[(Z)—N′-hydroxycarbamimidoyl]phenyl]methylcarbamoyl]azetidin-1-yl]-2-oxoethyl]amino]acetate) or biologically active analogs thereof; dabigatran (3-[[2-[(4-carbamimidoylanilino)methyl]-1-methylbenzimidazole-5-carbonyl]-pyridin-2-ylamino]propanoic acid) or biologically active analogs thereof such as dabigatran etexilate (ethyl 3-[[2-[[4-[(Z)—N′-hexoxycarbonylcarbamimidoyl]anilino]methyl]-1-methylbenzimidazole-5-carbonyl]-pyridin-2-ylamino]propanoate); RWJ-671818 (1-{N-[2-(amidinoaminooxy)ethyl]amino}carbonylmethyl-6-methyl-3-[2,2-difluoro-2-phenylethylamino]pyrazinone) or biologically active analogs thereof; 3-(2-phenethylamino)-6-methyl-1-(2-amino-6-methyl-5-methylenecarboxamidomethylpyridinyl)pyrazinone or biologically active analogs thereof; (E)-N-(3-((1-(benzo[b]thiophen-2-ylmethyl)-1H-1,2,3-triazol-4-yl)methoxy)phenyl)-2-(3-chlorophenyl)ethenesulfonamide or biologically active analogs thereof; and compound 2 by Merck ((S)—N-(2-(aminomethyl)-5-chlorobenzyl)-1-((R)-2-hydroxy-3,3-dimethylbutanoyl)pyrrolidine-2-carboxamide) or biologically active analogs thereof. Preferably, the direct thrombin inhibitor is an univalent direct thrombin inhibitor, preferably a small molecule suitable for oral administration, such as argatroban, melagatran and its prodrug ximelagatran, or dabigatran and its prodrug dabigatran etexilate, or biologically active analogs of these molecules.

Alternatively, the direct thrombin inhibitor is a peptide or peptidomimetic inhibitor (Mehta et al., 2014. Expert Opin Ther Pat 24: 47-67).

The term “coagulation factor XIa inhibitor”, as used herein, refers to inhibitors that are able to bind to the active site of coagulation factor XIa and inhibit its protease activity. The term includes direct coagulation factor XIa inhibitors, preferably small molecules, that bind to the active site of coagulation factor XIa and inhibit its protease activity. The group of direct coagulation factor XIa inhibitors include 4, 5, 6-trisubstituted pyrimidine derivatives; BMS-262084 ((2S,3R)-1-[4-(tert-butylcarbamoyl)piperazine-1-carbonyl]-3-[3-(diaminomethylideneamino)propyl]-4-oxoazetidine-2-carboxylic acid) or biologically active analogs thereof; compounds 1 (3′-[(2S,4R)-6-carbamimidoyl-4-methyl-4-phenyl-1,2,3,4-tetrahydroquinolin-2-yl]-4-carbamoyl-5′-[(3-methylbutanoyl)amino]biphenyl-2-carboxylic acid), 2 (trans-N—((S)-1-(4-(3-amino-1H-indazol-6-yl)-5-chloro-1H-imidazol-2-yl)-2-phenylethyl)-4-(aminomethyl)cyclohexanecarboxamide) and 33 ((2E)-N-{(1S)-1-[4-(3-amino-1H-indazol-6-yl)-1H-imidazol-2-yl]-2-phenylethyl}-3-[5-chloro-2-(1H-tetrazol-1-yl)phenyl]prop-2-enamide) of Bristol-Meyers Squibb, or biologically active analogs thereof; compound 13 of AstraZeneca (N-[(1S)-1-benzyl-2-[(6-chloro-2-oxo-1H-quinolin-4-yl)methylamino]-2-oxo-ethyl]-4-hydroxy-2-oxo-1H-quinoline-6-carbo) or biologically active analogs thereof; aryl boronic acids; macrocyclic indoles; and peptide or peptidomimetic inhibitors. Preferably, the coagulation factor XIa inhibitor is a direct coagulation factor XIa inhibitor, preferably a small molecule, that binds to the active site of coagulation factor XIa and inhibits its protease activity, preferably a small molecule suitable for oral administration.

The term “urokinase-type plasminogen activator inhibitor”, as used herein, refers to inhibitors that are able to bind to the active site of urokinase-type plasminogen activator and inhibit its protease activity. The term includes direct urokinase-type plasminogen activator inhibitors, preferably small molecules, that bind to the active site of urokinase-type plasminogen activator and inhibit its protease activity. The group of direct urokinase-type plasminogen activator inhibitors include, among others, WX-UK1 (ethyl 4-[(2S)-3-(3-carbamimidoylphenyl)-2-[[2,4,6-tri(propan-2-yl)phenyl]sulfonylamino]propanoyl]piperazine-1-carboxylate)—or its prodrug Upamostat, also known as Mesupron or WX-671—(the latter: Nα-(2,4,6-triisopropylphenylsulfonyl)-3-amidino-(L)-phenylalanine-4-ethoxycarbonylpiperazide), APC-10302 (6-chloro-2-(2-hydroxy-biphenyl-3-yl)-1H-indole-5-carboxamidine) or biologically active analogs of these compounds. Alternatively, the urokinase-type plasminogen activator inhibitor is a peptide or peptidomimetic inhibitor. Preferably, the direct urokinase-type plasminogen activator is an univalent direct urokinase-type plasminogen activator inhibitor, preferably a small molecule suitable for oral administration, such as WX-UK1 and its prodrug Upamostat.

The term “trypsin inhibitor”, as used herein, refers to an inhibitor that is able to bind to the active site of trypsin and inhibits its protease activity. The term includes a direct trypsin inhibitor, preferably a small molecule, that binds to the active site of trypsin and inhibit its protease activity. The group of direct trypsin inhibitors includes melagatran and its prodrug ximelagatran (the latter: ethyl 2-[[(1R)-1-cyclohexyl-2-[(2S)-2-[[4-[(Z)—N′-hydroxycarbamimidoyl]phenyl]methylcarbamoyl]azetidin-1-yl]-2-oxoethyl]amino]acetate), APC-10302 (6-chloro-2-(2-hydroxy-biphenyl-3-yl)-1H-indole-5-carboxamidine), and biologically active analogs of these molecules.

The term “biologically active analog” or “analog”, as used herein, refers to a derivative or a fragment of an indicated reference compound that exhibits the same biological function as the reference compound and retain the desired activity, such as an anti-coagulant or coagulant effect. The analog is thus preferably a structural and functional analog of the indicated reference compound.

The term “homologous”, as used herein, refers to the amino acid sequence identity between two amino acid sequences, expressed as a percentage of amino acid residues that are identical upon comparison of two amino acid sequences. Said comparison preferably is performed over the total length of the two amino acid sequences.

The term “region”, as used herein, refers to a stretch of amino acid residues that is bordered by two conserved amino acid residues. The region includes the two amino acids that border the region. The numbering of amino acid residues as applied herein is based on the amino acid sequences of SEQ ID NO: 1, SEQ ID NO: 2, SEQ ID NO: 3 and SEQ ID NO: 4. The term “region of amino acid”, as used herein, includes regions of amino acid residues corresponding to said defined regions, for instance regions in non-human thrombin, coagulation factor XIa, trypsin and/or urokinase-type plasminogen activator. The skilled person will understand that position of the region, for example in a non-human thrombin, might differ from the position in human thrombin as is indicated in SEQ ID NO: 1. However, the indicated region is flanked by two conserved amino acid residues which will allow the skilled person to identify a region corresponding to said defined region in a non-human protein.

The term “insertion” or “inserted”, as used herein, refers to the addition of at least one amino acid residue in a specific region of a native serine protease polypeptide, thereby increasing the number of amino acid residues in said region, compared to the number of amino acid residues in that region of the native serine protease polypeptide.

The term “replacement” or “replaced”, as used herein, refers to the substitution of one or more amino acid residues in a specific region, or at a specific site, of a serine protease polypeptide, thereby altering the amino acid sequence, but not the number of amino acid residues, in said region. A replacement is the consequence of the deletion of an amino acid residue followed by the insertion of a different amino acid residue at the same position.

The term “deletion” or “deleted”, as used herein, refers to the deletion of one or more amino acid residues in a specific region, or at a specific site, of a serine protease polypeptide, thereby reducing the number of amino acid residues in said region of said polypeptide compared to the number of amino acid residues in that region of the native serine protease polypeptide.

The term “native serine protease polypeptide”, as used herein, refers to an endogenous serine protease polypeptide that naturally occurs in an animal, preferably in a mammal, more preferably in a primate, more preferably in a human.

The term “amino acid composition”, as used herein, refers to the amino acid sequence and length of a stretch of amino acid residues, wherein the length is determined by the number of amino acid residues in that stretch.

The insertion, replacement and/or deletion, preferably insertion, of one or more amino acid residues can be performed using recombinant DNA techniques that are well known to the person skilled in the art. For example, the person skilled in the art can use synthetic DNA, PCR technology and molecular cloning to obtain recombinant DNA constructs having a DNA sequence encoding a protein of the present invention. Suitable methods and means are described in Green and Sambrook, “Molecular Cloning: A Laboratory Manual”, CSHL Press, 2012.

The term “outer-surface peptide structure”, as used herein, refers to a continuous stretch of amino acid residues, also referred to as a peptide loop or coil, appearing on the exterior of folded native thrombin, coagulation factor XIa, trypsin and urokinase-type plasminogen activator polypeptides. Preferably, the peptide structure is a region of amino acid residues corresponding to the region of amino acid residues between Gly-427 and Asp-462, preferably between His-450 and Asp-462, more preferably between His-450 and Leu-459, of SEQ ID NO: 1 for thrombin; a region of amino acid residues corresponding to the region of amino acid residues between Val-463 and Asp-480, preferably between His-469 and Asp-480 or Ser-477, of SEQ ID NO: 2 for coagulation factor XIa; a region of amino acid residues corresponding to the region of amino acid residues between Leu-73 and Asp-107, preferably between His-96 and Asp-107 or Leu-104, of SEQ ID NO: 3 for trypsin; and a region of amino acid residues corresponding to the region of amino acid residues between Val-237 and Asp-275, preferably between Phe-254 or Val-256 and Asp-275 or Asn-274, more preferably between His-262 and Asp-275 or Asn-274, of SEQ ID NO:4 for urokinase-type plasminogen activator.

It was found that insertion of at least one amino acid residue in said outer surface peptide structure, preferably a region of a thrombin, coagulation factor Xia, trypsin and urokinase-type plasminogen activator polypeptide as indicated hereinbefore and displayed in FIGS. 2-4, results in a protein having a decreased sensitivity to inhibition by serine protease inhibitors, preferably direct thrombin, coagulation factor XIa, trypsin or urokinase-type plasminogen activator inhibitors.

The phrase “corresponding to the region of amino acid residues between”, for example with regard to the region of amino acid residues corresponding to the region of amino acid residues between His-450 and Asp-462 or Leu-459 of SEQ ID NO: 1, is used herein to indicate that the residue number of the conserved His and Asp residues of another thrombin corresponding to the His-450 and Asp-462 or Leu-459 of SEQ ID NO: 1, may differ from the residue number attributed to said His and Asp residue in SEQ ID NO: 1. Differences in amino acid residue number can for example be the result of a different way of numbering amino acid residues. Also, and by way example, a difference in amino acid residue number can be the result of a difference in length of a prothrombin polypeptide as compared to the length of the human prothrombin polypeptide that is indicated in SEQ ID NO:1. Similarly, the amino acid residue Gly-427 of SEQ ID NO: 1 is conserved between prothrombin polypeptides of different species, as one skilled in the art would readily acknowledge when aligning multiple prothrombin polypeptides of different species. It is therefore possible to identify amino acid residues that correspond to said amino acid residues in a serine protease of a different species. The person skilled in the art will therefore understand that the amino acid residue numbering as applied herein is not limiting for the invention, but is only applied for clarity purposes.

The skilled person will know how to identify a region of amino acid residues that corresponds to the region of amino acid residues between said conserved amino acid residues of SEQ ID NO: 1, SEQ ID NO:2, SEQ ID NO:3 or SEQ ID NO:4 that border a region as described herein. The skilled person will, for example, directly establish that His-450 and Asp-462 of SEQ ID NO: 1 are highly conserved residues that are also present in serine protease polypeptides of other species.

Due to the highly conserved nature of the region of amino acid residues in and around His-450 and Asp-462 of SEQ ID NO: 1, or in and around the corresponding His and Asp residues in a non-human serine protease polypeptide, the person skilled in the art is able to identify a region of amino acid residues corresponding to the region of amino acid residues between His-450 and Asp-462 of SEQ ID NO: 1. The same general principle applies to other amino acid residues that border a region as described herein. In other words, the conserved nature of specific amino acid residues will give the skilled person an unambiguous pointer as to which amino acid residues are included in a region as defined herein in a non-human serine protease polypeptide. Amino acid residues that border a region, as specifically described herein, were found to be conserved between species and thus suitable to define a region.

A person skilled in the art will understand that the present invention inter alfa relates to the amino acid composition of a region of amino acid residues corresponding to the region of amino acid residues between (i) Gly-427 and Asp-462, preferably between His-450 and Asp-462, more preferably between His-450 and Leu-459, of SEQ ID NO: 1 for thrombin, (ii) between Val-463 and Asp-480, preferably between His-469 and Asp-480 or Ser-477, of SEQ ID NO: 2 for coagulation factor XIa, (iii) Leu-73 and Asp-107, preferably between His-96 and Asp-107 or Leu-104, of SEQ ID NO: 3 for trypsin and (iv) Val-237 and Asp-275, preferably between Phe-254 or Val-256 and Asp-275 or Asn-274, more preferably between His-262 and Asp-275 or Asn-274, of SEQ ID NO:4 for urokinase-type plasminogen activator. Therefore, the person skilled in the art will understand that the amino acid sequence of the remainder of a protein of the invention can vary, under the condition that said protein remains a, or can be activated into a, serine protease polypeptide with decreased sensitivity to serine protease inhibitors, preferably direct thrombin-, coagulation factor XIa-, trypsin-, or urokinase-type plasminogen activator inhibitors. Said remainder of a protein of the invention may thus vary as it for example varies between serine protease polypeptides of different species.

The number of amino acid residues in a region corresponding to the regions according to the invention, are conserved between serine protease polypeptides of different species, especially between species belonging to the group of mammals or to the group of primates. Hence, the number of amino acid residues is also conserved in serine protease polypeptides. Said conserved number of amino acid residues in a region of amino acid residues corresponding to the region between Gly-427 and Asp-462 of SEQ ID NO: 1 is 34, not including Gly-427 and Asp-462. Said conserved number of amino acid residues in a region of amino acid residues corresponding to the region between His-450 and Asp-462 of SEQ ID NO: 1 is 11, not including His-450 and Asp-462. For SEQ ID NOs 2, 3 and 4, the same principle applies.

It was found that the insertion of at least one amino acid residue in a region of amino acid residues corresponding to a region as defined herein in a protein of the invention, yields a catalytically active serine protease polypeptide albeit with decreased sensitivity to inhibition by serine protease inhibitors.

Preferably, the insertion is combined with a replacement of at least one amino acid residue in a region of amino acid residues as defined herein.

Particularly preferred is a protein of the invention wherein the insertion comprises 1-50, preferably 1-40, more preferably 1-30, and most preferably 1-20 amino acid residues. The insertion preferably comprises, or consists of, at least 1, 2, 3, 4, 5, 6, 7, 8, 9, 10, 11, 12, 13, 14, 15, 16, 17, 18, 19, or 20, more preferably 4 or 5, amino acid residues in a region of amino acid residues corresponding to the region between His-450 and Asp-462 or Leu-459 of SEQ ID NO: 1 for thrombin, His-469 and Asp-480 or Ser-477 of SEQ ID NO: 2 for coagulation factor XIa, His-96 and Asp-107 or Leu-104 of SEQ ID NO: 3 for trypsin, or His-262 and Asp-275, Asn-274 or Ala-271 of SEQ ID NO: 4 for urokinase-type plasminogen activator. Particularly preferred is the insertion of at least 4 amino acid residues, such as an insertion of 8 amino acid residues in a region of amino acid residues corresponding to the region between His-450 and Asp-462 or Leu-459 of SEQ ID NO: 1 for thrombin. Also, particularly preferred is the insertion of at least 5 amino acid residues, such as an insertion of 9 amino acid residues in a region of amino acid residues corresponding to the region between His-469 and Asp-480 or Ser-477 of SEQ ID NO: 2 for coagulation factor XIa. Also, particularly preferred is the insertion of at least 5 amino acid residues, such as an insertion of 9 or 11 amino acid residues in a region of amino acid residues corresponding to the region between His-96 and Asp-107 or Leu-104 of SEQ ID NO: 3 for trypsin. Also, particularly preferred is the insertion of at least 3 amino acid residues, such as an insertion of 7 amino acid residues in a region of amino acid residues corresponding to the region between His-262 and Asp-275, Asn-274 or Ala-271 of SEQ ID NO: 4 for urokinase-type plasminogen activator. The person skilled in the art will understand that the amino acid residues can be inserted at any position in a region of amino acid residues corresponding to a region of amino acid residues as defined herein. An amino acid residue suitable for insertion is selected from the group of twenty naturally occurring amino acid residues as listed in Table 1. The person skilled in the art will understand that said inserted amino acid residues may undergo a post-translational chemical alteration in vivo or in vitro. As is indicated herein above, the person skilled in the art can use synthetic DNA, PCR technology and molecular cloning to obtain recombinant DNA constructs having a DNA sequence encoding a protein of the present invention comprising an insertion of between 1-50 amino acid residues in a region of amino acid residues corresponding to a region as defined herein.

The insertion in a region of amino acid residues corresponding to the region of amino acid residues is preferably between Trp-455 and Arg-456 of SEQ ID NO: 1, for thrombin; between Met-474 and Ala-475 of SEQ ID NO: 2, for coagulation factor XIa; between Asp-100 and Arg-101 of SEQ ID NO: 3 for trypsin, between Thr-269 and Leu-270 of SEQ ID NO: 4, for urokinase-type plasminogen activator, or between two amino acid residues corresponding to these amino acid residues in a non-human thrombin, coagulation factor XIa, trypsin or urokinase-type plasminogen activator polypeptide.

Particularly preferred is a protein of the invention comprising an insertion of at least one amino acid residues, combined with a replacement of 1-30, preferably 1-8 amino acid residues. Said replacement preferably comprises, or consists of, 1, 2, 3, 4, 5, 8, 9, 10, 11, 12, 13, 14, 15, 16, 17, 18, 19, 20, 21, 22, 23, 24, 25, 26, 27, 28, 29 or 30, preferably 5-8 amino acid residues. The replacement of amino acid residues in a region of amino acid residues corresponding to a region of amino acid residues between His-450 and Asp-462 or Leu-459, of SEQ ID NO: 1, for thrombin, preferably comprises, or consists of, 7 or 8 amino acid residues. The replacement of amino acid residues in a region of amino acid residues corresponding to a region of amino acid residues between His-469 and Asp-480 or Ser-477, of SEQ ID NO: 2, for coagulation factor XIa, preferably comprises, or consists of, 5 or 6 amino acid residues. The replacement of amino acid residues in a region of amino acid residues corresponding to a region of amino acid residues between His-96 and Asp-107 or Leu-104, of SEQ ID NO: 3, for trypsin, preferably comprises, or consists of, 7 amino acid residues. The replacement of amino acid residues in a region of amino acid residues corresponding to a region of amino acid residues between His-262 and Asp-275, of SEQ ID NO: 4, for urokinase-type plasminogen activator, preferably comprises, or consists of, 4 or 7 amino acid residues.

An amino acid residue present in a region corresponding to a region as defined herein of a protein of the invention is preferably replaced by any one of the amino acid residues listed in Table 1, preferably by an amino acid of the same group as is indicated in the columns “side chain polarity” and “side chain charge” in Table 1. Preferably, one or more of amino acid residues 451-455 and 456-458 of SEQ ID NO:1; one or more of amino acid residues 470-474 and 475-476 of SEQ ID NO:2; one or more of amino acid residues 97-100 and 101-103 of SEQ ID NO:3; one or more of amino acid residues 262-269 and 270-274 of SEQ ID NO:4, or their corresponding amino acid residues in a non-human serine protease are replaced by a different amino acid residue selected from the amino acid residues as indicated in Table 1.

The person skilled in the art will understand that when amino acid residues are replaced in a region of amino acid residues corresponding to a region as defined herein of a non-human serine protease of the invention, only those amino acid residues are preferably replaced that are not already present in a preferred protein of the invention. The person skilled in the art will know that the aforementioned reference to SEQ ID NOs is only made in the context of exemplifying the replacement of amino acid residues in a specified region of amino acid residues. He will therefore have an indication which one or more amino acid residues may be replaced in a non-human serine protease for what other amino acid residue or residues. The invention is directed to all possible combinations of the aforementioned insertion and replacement.

A protein of the invention may further comprise a deletion of at least one amino acid residue in a region of amino acid residues corresponding to a region as defined herein, provided that the total number of amino acid residues in said region after the insertion of at least one amino acid residue and deletion of at least one amino acid residue is increased, when compared to the number of amino acid residues in that region of the native serine protease polypeptide. Particularly preferred is a protein of the invention having a deletion of at least 1, 2, 3, 4, 5, 6, 7, 8, 10, 15, 20 or 30 amino acid residues.

A preferred protein of the invention comprises a combination of an insertion and a replacement, or a combination of an insertion, a replacement, and/or a deletion. Insertions and deletions may occur independently of each other and it is thus possible that, for example, an insertion of 5 amino acid residues and a deletion of 4 amino acid are present at different amino acid positions in a region of amino acid residues corresponding to a region of amino acid residues as defined herein, thereby increasing the total number of amino acid residues in a serine protease polypeptide of the invention. The skilled person will understand that an insertion or deletion changes the amino acid residue numbering in a protein.

A protein of the invention most preferably comprises a region of amino acid residues having the amino acid sequence of SEQ ID NO: 5, SEQ ID NO: 6, SEQ ID NO: 13, SEQ ID NO: 14, SEQ ID NO: 15 or SEQ ID NO: 16 between amino acid residues His-450 and Asp-462 of SEQ ID NO:1, for thrombin, or between amino residues corresponding to His-450 and Asp-462 of SEQ ID NO:1, for non-human thrombin.

A protein of the invention most preferably comprises a region of amino acid residues having the amino acid sequence of SEQ ID NO: 7 or SEQ ID NO: 8 between amino acid residues His-469 and Asp-480 of SEQ ID NO:2, for coagulation factor XIa, or between amino residues corresponding to His-469 and Asp-480 of SEQ ID NO:2, for non-human coagulation factor XIa.

A protein of the invention most preferably comprises a region of amino acid residues having the amino acid sequence of SEQ ID NO: 9 or SEQ ID NO: 10 between amino acid residues His-96 and Asp-107 of SEQ ID NO:3, for trypsin, or between amino residues corresponding to His-96 and Asp-107 of SEQ ID NO:3, for non-human trypsin.

A protein of the invention most preferably comprises a region of amino acid residues having the amino acid sequence of SEQ ID NO: 11 or SEQ ID NO: 12 between amino acid residues His-262 and Asp-275 of SEQ ID NO:4, for urokinase-type plasminogen activator, or between amino residues corresponding to His-262 and Asp-275 of SEQ ID NO:4, for non-human urokinase-type plasminogen activator.

The present invention also encompasses proteins that are substantially homologous and biologically equivalent to a protein of the invention. A protein of the invention preferably has an amino acid sequence that is more than 90% homologous to SEQ ID NO: 1, SEQ ID NO: 2, SEQ ID NO: 3 or SEQ ID NO:4; or to the activated forms thereof, wherein said protein is catalytically active, or catalytically active after processing/activation, and has a decreased sensitivity to a serine protease inhibitor, preferably a direct thrombin-, coagulation FXIa, -trypsin-, or urokinase-type plasminogen activator inhibitor.

An alternative protein of the invention is a recombinant protein comprising a serine protease polypeptide having an amino acid residue replacement or substitution on an amino acid residue position corresponding to Ile-542 of SEQ ID NO:1, wherein the serine protease polypeptide is not a coagulation factor X polypeptide or naturally processed or activated forms thereof. For example, the skilled person is aware that position Ile-542 of SEQ ID NO:1 corresponds to position His-552 of SEQ ID NO:2, position Gly-177 of SEQ ID NO:3, and position Ser-353 of SEQ ID NO:4. The skilled person has no difficulty in identifying amino acid residue positions that correspond to Ile-542 of SEQ ID NO:1, His-552 of SEQ ID NO:2, Gly-177 of SEQ ID NO:3, or Ser-353 of SEQ ID NO:4 in alternative serine protease polypeptides. Preferably, the replacement is on amino acid residue position Ile-542 of SEQ ID NO:1, His-552 of SEQ ID NO:2, Gly-177 of SEQ ID NO:3, or Ser-353 of SEQ ID NO:4. The replacement or substitution is a mutation, preferably a conservative or non-conservative mutation. Preferably, the amino acid residue as replacement can be any one of the amino acid residues as indicated in Table 1. More preferably, the amino acid residues as replacement is an alanine, serine, phenylalanine or glutamic acid. Preferably, the serine protease polypeptide is a (pro)thrombin. The amino acid residue replacement in a protein as described in this section can be combined with any insertion described herein.

The term “decreased sensitivity to serine protease inhibitors”, or “decreased sensitivity to inhibition by serine protease inhibitors, as used in the context of the present invention, refers to the concentration of a serine protease inhibitor that is required to produce 50% of the maximum inhibition (Ki). This concentration is higher for a polypeptide of the present invention than for a native serine protease polypeptide. Said native serine protease polypeptide is preferably derived from blood plasma or is recombinantly produced. The Ki of a serine protease inhibitor is preferably determined by pre-incubating a protein of the invention with 0.001 to 100 μM of a serine protease inhibitor and subsequently performing an experiment wherein the catalytic activity is assayed.

The Ki of a protein of the invention is preferably more than 2× increased, more preferably between 50× and 100× increased, and most preferably more than 100× increased as compared to the Ki of said native serine protease polypeptide without an insertion of at least one amino acid residue in a region of amino acid residues corresponding to a region of amino as defined herein.

The invention further provides a nucleic acid molecule comprising a nucleotide sequence, preferably a DNA sequence, that encodes a protein of the invention. The person skilled in the art will understand how to generate a DNA sequence that encodes an amino acid sequence of a protein of the present invention and how to manufacture and isolate a nucleic acid molecule with said DNA sequence using generally known recombinant DNA techniques. The sequence of the nucleic acid molecule is preferably codon-optimized for expression in a host cell of the invention. In this way codons are used that are favored for high level expression in a specific host cell.

The present invention also provides an expression vector comprising a nucleic acid molecule of the invention.

Nucleic acid molecules are preferably inserted in an expression vector using recombinant DNA techniques known by the person skilled in the art. Expression vectors in the context of the invention direct the expression of a protein of the invention in a host cell. These expression vectors are preferably replicable in a host cell, either as episomes or as part of the chromosomal DNA. Further, the expression vector preferably comprises (i) a strong promoter/enhancer, such as the CMV or SV40 promoter, (ii) an optimal translation initiation sequence, such as a ribosomal binding site and start codon, preferably a KOZAK consensus sequence and (iii) a transcription termination sequence, including a poly(A) signal when the protein is expressed in eukaryotic cells. Suitable expression vectors include plasmids and viral vectors such as adenoviruses, adeno-associated viruses and retroviruses. The person skilled in the art will understand that the expression vector to be used is dependent on the host cell that is used for expression of a recombinant protein. An expression vector of the invention is preferably suited for expression of a nucleic acid molecule of the invention in a prokaryotic cell including a bacterial cell, or, more preferred, in a eukaryotic host cell, such as a yeast cell and a mammalian cell. Particularly preferred is mammalian expression vector pCMV4.

As an alternative, a nucleic acid molecule of the invention may be inserted in the genome of a host cell. Said insertion preferably is at a locus or within a region that ensures expression of a nucleic acid molecule of the invention in the host cell.

The invention further provides a host cell comprising a nucleic acid molecule or expression vector according to the invention. The invention preferably provides a host cell expressing a nucleic acid molecule of the invention thereby producing a protein of the invention. Said protein is either produced within the host cell or, preferably secreted from the host cell.

Suitable host cells for use in the present invention include prokaryotic and eukaryotic cells, such as bacterial cells, yeast cells, insect cells, animal cells, mammalian cells, murine cells, rat cells, sheep cells, simian cells and human cells. Examples of suitable eukaryotic host cells include, but are not limited to HEK 293 cells, the hamster cell line CHO and BHK-21; the murine host cells NIH3T3, NSO and C127; the simian host cells COS and Vero; and the human host cells HeLa, PER.C6, U-937 and Hep G2. Suitable cells are available from public sources such as ATCC and Life Technologies. A number of transfection techniques are known in the art, see, e.g., Graham et al., 1973. Virology 52: 456; Green et al., 2012. “Molecular Cloning: A Laboratory Manual”, CSHL Press; Davis et al., “Basic Methods in Molecular Biology”, 1986, Elsevier; and Chu et al., 1981. Gene 13: 197. The person skilled in the art preferably employs techniques as described in these references to introduce one or more exogenous nucleic acid molecules into suitable host cells.

A particularly preferred host cell for the production of a protein of the invention is a HEK 293 cell.

The invention further provides a pharmaceutical composition comprising a protein of the invention, and a pharmaceutically acceptable carrier or excipient. A pharmaceutical composition of the invention preferably comprises one or more of diluents, fillers, salts, buffers, stabilizers, solubilizers, and other materials known in the art. The characteristics of the carrier will depend on the route of administration, as is known to the skilled person. To reduce a potential thrombotic risk of administering an activated serine protease polypeptide, a pharmaceutical composition of the invention preferably comprises a protein of the invention that is activated after administrating to the subject.

The term “subject” refers to the group of mammals, preferably humans.

The term “pharmaceutical composition” refers, in the context of the invention, to a combination of a protein of the invention with a carrier, inert or active, making the composition suitable for therapeutic use in vivo or ex vivo.

The term “pharmaceutically acceptable”, as used herein, refers to a nontoxic material that is compatible with the physical and chemical characteristics of a protein of the invention and does not interfere with the effectiveness of the biological activity of said protein.

A pharmaceutical composition of the invention may be adapted for enteral administration of the composition, wherein the composition is absorbed through the digestive tract, e.g., oral ingestion or rectal administration. Said composition is preferably encapsulated, for example by liposomes, to prevent proteolytic degradation.

A pharmaceutical composition of the invention is preferably adapted for parenteral administration, wherein the composition is intravenously, intra-arterial, subcutaneously, and/or intramuscularly introduced. Parenteral administration involves the injection or infusion of a pharmaceutical composition of the invention into a body tissue or body fluid, whereby preferably a syringe, needle, or catheter is used. As an alternative, needle-less high-pressure administration may be used as means for parenteral administration.

For injectable compositions (e.g., intravenous compositions), the carrier may be an aqueous or oily solution, dispersions, emulsions and/or suspension. Preferably, the carrier is an aqueous solution, preferably distilled sterile water, saline, buffered saline, or another pharmaceutically acceptable excipient for injection.

A pharmaceutical composition of the invention, comprising a protein of the invention that is a thrombin or coagulation factor XIa, can be applied locally, for example at or in a wound or to a blood vessel, preferably an artery, that supplies the wounded region with blood. Said local administration is a topical administration, for example in the form of a cream, foam, gel, lotion or ointment, or a parenteral administration, for example by injection or infusion, to generate a local or systemic therapeutic effect. Topical administration of a protein of the invention for a local effect reduces the risk of a potential systemic thrombotic incident.

A pharmaceutical composition of the invention is preferably used in a variety of therapeutical applications. For example, the pharmaceutical composition comprising a protein of the invention that is a thrombin or coagulation factor XIa, can be used as bypassing agent in the treatment or amelioration of disorders wherein normal blood coagulation is impaired, such as in hemophilia A and B, including in hemophilia A and B inhibitor patient groups. A hemophilia A and B inhibitor patient is a patient that has develop an antibody that is directed against he product used to treat or prevent bleeding episodes.

The invention thus also provides a protein or pharmaceutical composition according to the invention for use as a medicament.

The invention further provides a protein according to the invention that comprises a thrombin or coagulation factor XIa polypeptide according to the invention, or a pharmaceutical composition according to the invention comprising a thrombin or coagulation factor XIa polypeptide, for use in a method of completely or partially reversing an anti-coagulant effect of a coagulation inhibitor in a subject.

The term “anti-coagulant effect” refers to the therapeutic effect, such as the prevention of blood clotting, that is the result of the action of coagulation inhibitors.

The invention further provides the use of a protein of the invention comprising a thrombin or coagulation factor XIa polypeptide, for the manufacture of a medicament for completely or partially reversing an anti-coagulant effect of a coagulation inhibitor in a subject.

The invention further provides a method of completely or partially reverting an anti-coagulant effect of a coagulation inhibitor in a subject, said method comprising administering to said subject a therapeutically effective amount of a protein of the invention comprising a thrombin or coagulation factor XIa polypeptide, or a pharmaceutical composition of the invention comprising a thrombin or coagulation factor XIa polypeptide. Preferably, a method of the invention is applied for preventing or ameliorating bleeding complications that are associated with anticoagulant therapy.

The term “therapeutically effective amount” as used herein, means that the amount of the active ingredient contained in the pharmaceutical composition to be administered is of sufficient quantity to achieve the intended purpose, such as, inter alia in this case, to completely or partially reverse an anti-coagulant effect of a coagulation inhibitor. The amount of active ingredient, i.e. a protein of the invention, in a pharmaceutical composition according to the invention preferably is in the range of about 5 mg to 10 gram of protein.

A therapeutically effective amount may depend on the average concentration of a protein in the blood of a person in need of a protein of the invention. For example, a therapeutically effective amount (i) preferably is between 5 mg and 10 g, preferably between 150 mg to 10 gram of a thrombin according to the invention; (ii) 5 mg to 600 mg, preferably 5 mg to 300 mg, of a coagulation factor XIa according to the invention; (iii) 100 microgram to 7 mg of a trypsin according to the invention; and (iv) 0.004 mg to 0.3 mg of a urokinase-type plasminogen activator according to the invention. The skilled person understands that the dosage of each of the proteins according to the invention may differ, since normal plasma or serum levels of these protein differ.

A pharmaceutical composition according to the invention comprising a protein of the invention comprising a thrombin or coagulation factor XIa polypeptide of the invention is preferably administered only once, twice or three times, preferably only once, to a subject in need of complete or partial reversal of an anti-coagulant effect of a coagulation inhibitor.

The invention further provides a protein of the invention comprising a trypsin polypeptide of the invention, or a pharmaceutical composition of the invention comprising a protein of the invention comprising a trypsin polypeptide of the invention, for use in a method of completely or partially reversing the inhibition of peptide bond hydrolysis of a trypsin inhibitor in a subject.

In the same context, the invention provides a method of completely or partially reversing the inhibition of peptide bond hydrolysis of a trypsin inhibitor in a subject, said method comprising administering to said subject a therapeutically effective amount of a protein of the invention comprising a trypsin polypeptide of the invention, or a pharmaceutical composition of the invention comprising a protein of the invention comprising a trypsin polypeptide of the invention. Preferably, the subject is treated with a trypsin inhibitor before administering a protein of the invention.

In the same context, the invention provides the use of a protein of the invention comprising a trypsin polypeptide according to the invention for the manufacture of a medicament for completely or partially reversing the inhibition of peptide bond hydrolysis of a trypsin inhibitor in a subject.

The invention further provides a non-therapeutic use of a protein of the invention comprising a trypsin polypeptide, in completely or partially reversing the inhibition of peptide bond hydrolysis of a trypsin inhibitor.

The invention further provides a protein of the invention comprising a urokinase-type plasminogen activator of the invention, for use in completely or partially reversing an anti-fibrinolytic effect, preferably said anti-fibrinolytic effect being induced by an inhibitor such as a urokinase-type plasminogen activator inhibitor, in a subject. Preferably, in this context, the anti-fibrinolytic effect results in the decreased break down of a blood clot, preferably in the context of thrombosis such as severe or massive deep venous thrombosis, pulmonary embolism, myocardial infarction or occluded intravenous or dialysis cannulas. It is further preferred that the urokinase-type plasminogen activator inhibitor is Mesupron.

The subject preferably is a cancer patient treated with Mesupron. An urokinase-type plasminogen activator inhibitor such as Mesupron is often administered to a cancer patient to prevent tissue degradation, which would contribute to metastasis. However, the administration of an urokinase-type plasminogen activator inhibitor such as Mesupron may result in an anti-fibrinolytic effect, which can be treated with a recombinant urokinase-type plasminogen activator according to the invention.

In addition to other routes of administration as described herein, the urokinase-type plasminogen activator of the invention can be formulated for intrapleural administration, for example so as to improve the drainage of complicated pleural effusions and empyemas.

In the same manner, the invention provides a protein of the invention comprising a urokinase-type plasminogen activator of the invention for the manufacture of a medicament for completely or partially reversing an anti-fibrinolytic effect, preferably said anti-fibrinolytic effect being induced by an inhibitor such as a urokinase-type plasminogen activator inhibitor, in a subject.

In the same manner, the invention provides a method of completely or partially reverting an anti-fibrinolytic effect, preferably said anti-fibrinolytic effect being induced by an inhibitor such as a urokinase-type plasminogen activator inhibitor, in a subject, said method comprising administering to said subject a therapeutically effective amount of a protein of the invention comprising a urokinase-type plasminogen activator polypeptide, or a pharmaceutical composition of the invention comprising a urokinase-type plasminogen activator polypeptide. Preferably, a method of the invention is applied for preventing or ameliorating thrombotic complications that are associated with anti-fibrinolytic-inhibitor therapy.

Alternatively, the invention provides a recombinant protein comprising a serine protease polypeptide, said polypeptide comprising an insertion of at least one amino acid residue in an outer-surface peptide structure, wherein the serine protease polypeptide is not a coagulation factor X polypeptide, or catalytically active or naturally processed forms thereof such as a coagulation factor Xa polypeptide.

Preferably, said peptide structure is a region of amino acid residues corresponding to the region of amino acid residues between His-450 and Asp-462 of SEQ ID NO: 1. In this context, the term “corresponding” is used to refer to regions of amino acid residues in thrombin and non-thrombin serine proteases. Alternatively, the serine protease polypeptide is selected from the group consisting of thrombin, coagulation factor XIa, trypsin and urokinase-type plasminogen activator, wherein the outer-surface peptide structure are as indicated herein.

In the context of the invention, a protein is a coagulation FX or FXa polypeptide if it is a (potentially) procoagulant serine protease and if the full-length amino acid sequence of said protein comprises stretches of, or single, amino acid residues that correspond to stretches of, or single, amino acid residues that are conserved between coagulation FX factors of different species. For example, a procoagulant serine protease comprising a polypeptide that contains stretches of amino acid residues that correspond to amino acid residues Cys-246 to Ala-250, Phe-260 to Leu-266 and/or Asp-413 to His-423 of human coagulation factor X (vide Genbank Acc No. AAH46125.1), is assumed to be a coagulation FXa polypeptide. As stated hereinbefore, the indicated His and Asp amino acid residues are conserved between different serine protease polypeptides and between different species.

For the purpose of clarity and a concise description, features are described herein as part of the same or separate embodiments, however, it will be appreciated that the scope of the invention may include embodiments having combinations of all or some of the features described.

(human coagulation prothrombin protein)
SEQ ID NO: 1

| | |
|---|---|
| 1 | mahvrglqlp gclalaalcs lvhsqhvfla pqqarsllqr vrrantflee vrkgnlerec |
| 61 | veetcsyeea fealesstat dvfwakytac etartprdkl aaclegncae glgtnyrghv |
| 121 | nitrsgiecq lwrsryphkp einstthpga dlqenfcrnp dssttgpwcy ttdptvrrqe |
| 181 | csipvcgqdq vtvamtprse gssvnlsppl eqcvpdrgqq yqgrlavtth glpclawasa |
| 241 | qakalskhqd fnsavqlven fcrnpdgdee gvwcyvagkp gdfgycdlny ceeaveeetg |
| 301 | dgldedsdra iegrtatsey qtffnprtfg sgeadcglrp lfekksledk terellesyi |
| 361 | dgrivegsda eigmspwqvm lfrkspqell cgaslisdrw vltaahclly ppwdknften |
| 421 | dllvrigkhs rtryerniek ismlekiyih prynwrenld rdialmklkk pvafsdyihp |
| 481 | vclpdretaa sllqagykgr vtgwgnlket wtanvgkgqp svlqvvnlpi verpvckdst |
| 541 | riritdnmfc agykpdegkr gdacegdsgg pfvmkspfnn rwyqmgivsw gegcdrdgky |
| 601 | gfythvfrlk kwiqkvidqf ge |

(Human coagulation factor XI protein)
SEQ ID NO: 2

| | |
|---|---|
| 1 | miflyqvvhf ilftsvsgec vtqllkdtcf eggdittvft psakycqvvc tyhprcllft |
| 61 | ftaespsedp trwftcvlkd svtetlprvn rtaaisgysf kqcshqisac nkdiyvdldm |
| 121 | kginynssva ksagecqerc tddvhchfft yatrqfpsle hrnicllkht qtgtptritk |
| 181 | ldkvvsgfsl kscalsnlac irdifpntvf adsnidsvma pdafvcgric thhpgclfft |
| 241 | ffsqewpkes qrnlcllkts esglpstrik kskalsgfsl qscrhsipvf chssfyhdtd |
| 301 | flgeeldiva aksheacqkl ctnavrcqff tytpaqascn egkgkcylkl ssngsptkil |
| 361 | hgrggisgyt lrlckmdnec ttkikprivg gtasvrgewp wqvtlhttsp tqrhlcggsi |
| 421 | ignqwiltaa hcfygvespk ilrvysgiln qseikedtsf fgvqeiiihd qykmaesgyd |
| 481 | iallklettv nytdsqrpic lpskgdrnvi ytdcwvtgwg yrklrdkiqn tlqkakiplv |
| 541 | tneecqkryr ghkithkmic agyreggkda ckgdsggpls ckhnevwhlv gitswgegca |
| 601 | qrerpgvytn vveyvdwile ktqav |

(Human trypsin-1 protein)
SEQ ID NO: 3

| | |
|---|---|
| 1 | mnplliltfv aaalaapfdd ddkivggync eensvpyqvs lnsgyhfcgg slineqwvvs |
| 61 | aghcyksriq vrlgehniev legneqfina akiirhpqyd rktlnndiml iklssravin |
| 121 | arvstislpt appatgtkcl isgwgntass gadypdelqc ldapvlsgak ceasypgkit |
| 181 | snmfcvgfle ggkdscqgds ggpvvongql qgvvswgdgc aqknkpgvyt kvynyvkwik |
| 241 | ntiaans |

(Human urokinase-type plasminogen activator)
SEQ ID NO: 4

| | |
|---|---|
| 1 | mrallarlll cvlvvsdskg snelhqvpsn cdclnggtcv snkyfsnihw cncpkkfggq |
| 61 | hceidksktc yegnghfyrg kastdtmgrp clpwnsatvl qqtyhahrsd alqlglgkhn |
| 121 | ycrnpdnrrr pwcyvqvglk plvqecmvhd cadgkkpssp peelkfqcgq ktlrprfkii |
| 181 | ggefttienq pwfaaiyrrh rggsvtyvcg gslispcwvi sathcfidyp kkedyivylg |
| 241 | rsrlnsntqg emkfevenli lhkdysadtl ahhndiallk irskegrcaq psrtigticl |
| 301 | psmyndpqfg tsceitgfgk enstdylype qlkmtvvkli shrecqqphy ygsevttkml |
| 361 | caadpqwktd scqgdsggpl vcslqgrmtl tgivswgrgc alkdkpgvyt rvshflpwir |
| 421 | shtkeengla l |

SEQ ID NO: 5

| | |
|---|---|
| 1 | kkfvppqkay kfdlaaldr |

SEQ ID NO: 6

| | |
|---|---|
| 1 | ppqkaykfdl aaldr |

SEQ ID NO: 7

| | |
|---|---|
| 1 | kkfvppqkay kfdlaasgy |

SEQ ID NO: 8

| | |
|---|---|
| 1 | ppqkaykfdl aasgy |

SEQ ID NO: 9

| | |
|---|---|
| 1 | kkfvppqkay kfdlaalnn |

SEQ ID NO: 10

| | |
|---|---|
| 1 | kkfvppsqef yekfdlvsln n |

SEQ ID NO: 11

| | |
|---|---|
| 1 | kkfvppqkay kfdlaahhn |

SEQ ID NO: 12

| | |
|---|---|
| 1 | ppqkaykfdl aahhn |

SEQ ID NO: 13

| | |
|---|---|
| 1 | pryvppqkay kfdlaaldr |

SEQ ID NO: 14

| | |
|---|---|
| 1 | tkfvppnyyy vhqnfdrval dr |

SEQ ID NO: 15

| | |
|---|---|
| 1 | pkyhqgsgpi lprrtldr |

SEQ ID NO: 16

| | |
|---|---|
| 1 | prydsissky lkellekpld r |

### FIGURE LEGENDS

FIG. 1. The top panel displays the alignment of the region of amino acid residues from His-450 to Asp-462 in human prothrombin (SEQ ID NO: 1) with the region of amino acid residues corresponding to said region in human FXI (SEQ ID NO: 2), human trypsin (SEQ ID NO: 3) and human urokinase-type plasminogen activator (SEQ ID NO:4). The lower three panels display the newly generated ‘A’ and ‘B’ protein variants of human prothrombin, human FXI, human trypsin and human urokinase-type plasminogen activator having an insertion in the region of amino acid residues corresponding to said region of His-450 to Asp-462 in human prothrombin, between His-469 and Asp-480 for human FXI, between His-96 and Asp-107 for trypsin and between His-262 and Asp-275 for urokinase-type plasminogen activator. The conserved residues histidine and aspartic acid are highlighted.

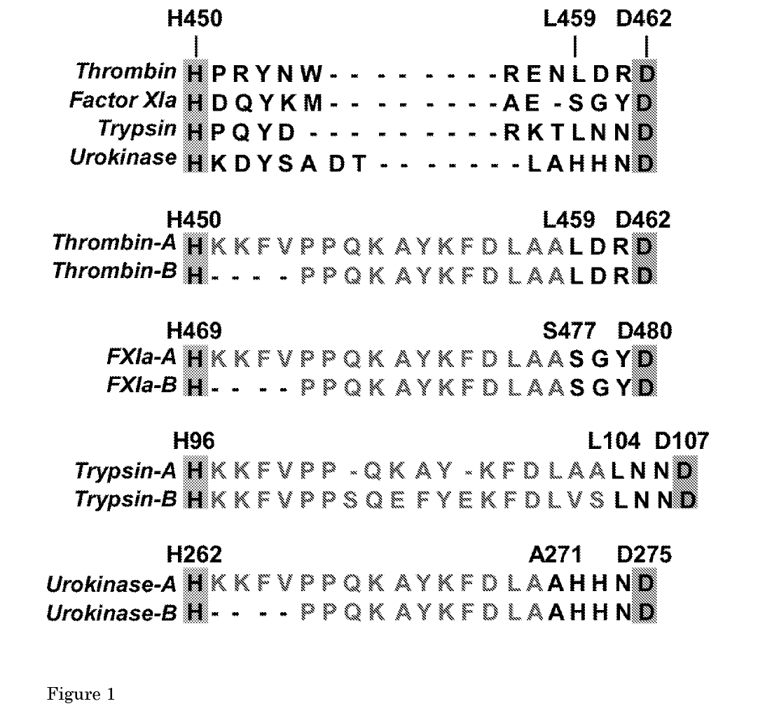

FIG. 2. The crystal structure of the argatroban-thrombin complex is shown (PDB 1DWC). The argatroban-contact residues (His57, Tyr60A, Lys60F, Leu99, Ile174, Glu192, Ser195, Asp189, Glu192, Gly216, Gly218, Gly226; chymotrypsin numbering (Bode, W. et al. 1989. EMBO J 8: 3467-3475)) and catalytic triad residues His57, Ser195, and Asp102 are shown as sticks. The His91 loop is highlighted and indicated with an arrow.

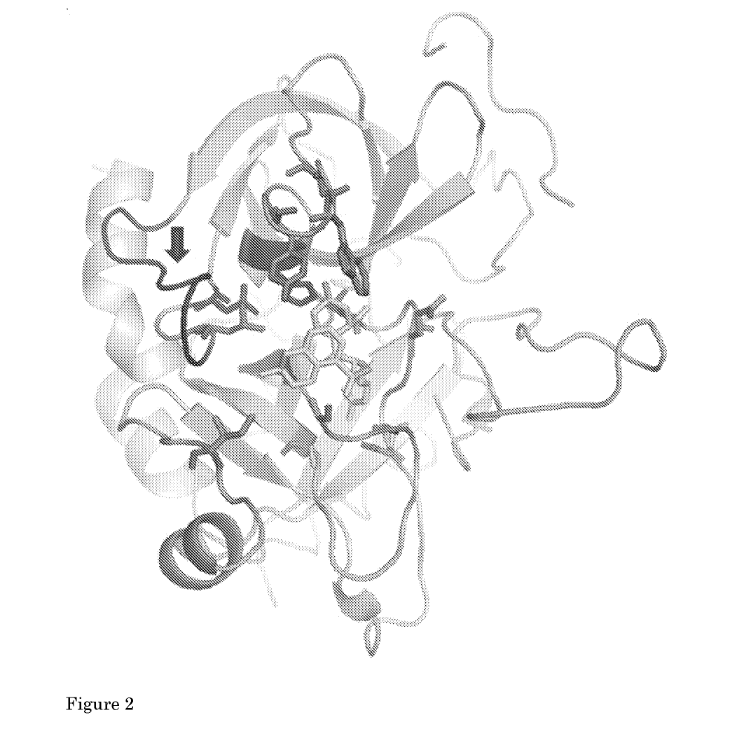

FIG. 3. The crystal structure of the compound 33-FXIa complex is shown (PDB 4X6P). Compound 33 is shown in a stick model, FXIa is shown in surface representation. The contact residues (His40, Leu41, Cys42, Cys58, Tyr58b, Tyr143, Ile151, Asp189, Lys192, Gly193, Asp 194, Ser195, Gly216, Gly218, Tyr228; chymotrypsin numbering (Bode, W. et al. 1989. EMBO J 8: 3467-3475)), the catalytic triad residues His57, Ser195, and the His91 loop are highlighted. The latter is additionally indicated with an arrow.

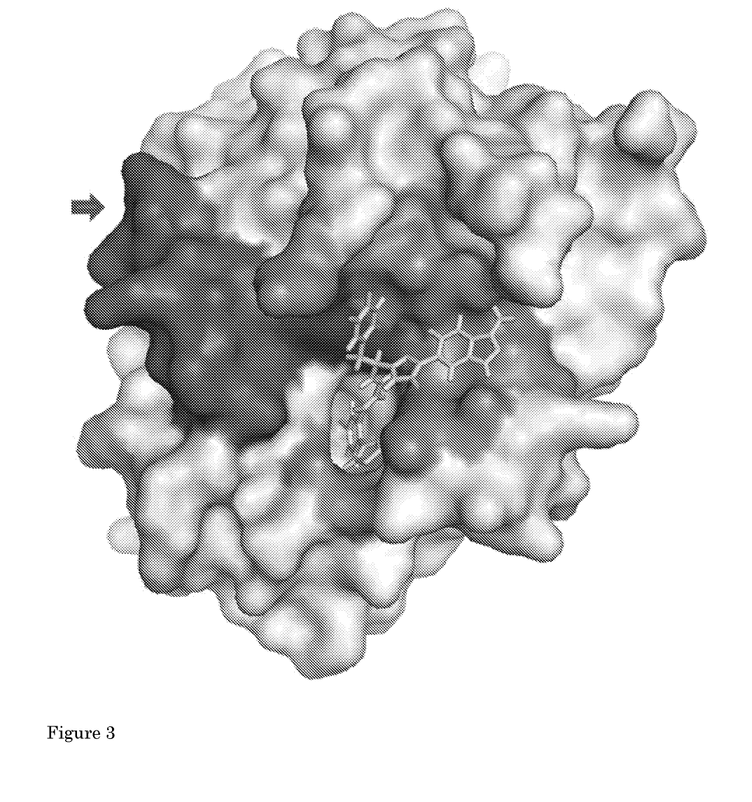

FIG. 4. The crystal structure of the melagatran-trypsin complex is shown (PDB 1K1P). The contact residues (Asp 189, Ser190, Gly216, and Gly21; chymotrypsin numbering (Bode, W. et al. 1989. EMBO J 8: 3467-3475)) and the catalytic triad residues His57, Ser195 are shown as sticks. The His91 loop is highlighted and indicated with an arrow.

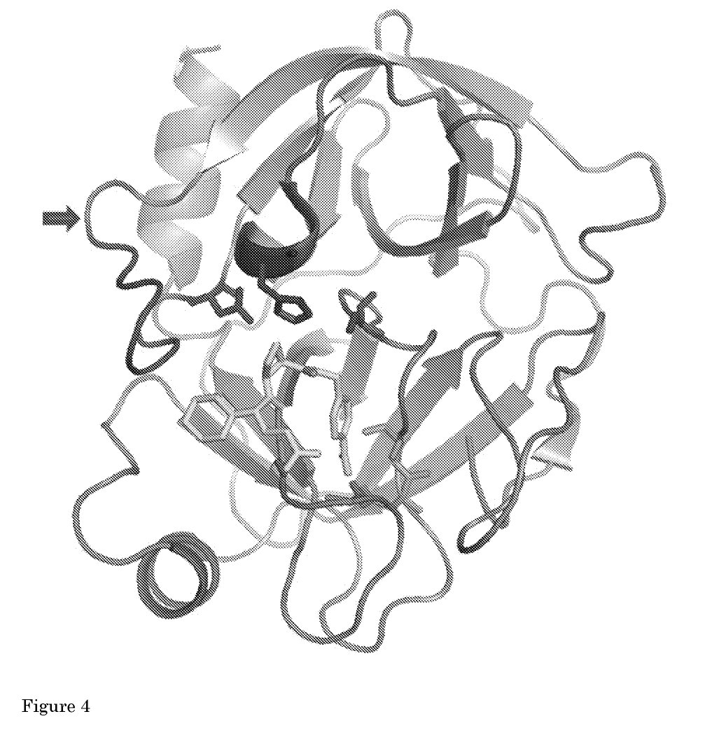

FIG. 5. An alignment of the region of amino acid residues from His-450 to Asp-462 in human prothrombin (‘Thrombin’, SEQ ID NO: 1) and the newly generated protein variants of human prothrombin (‘ISO1’, ‘ISO2’, ‘NSC’, ‘KL10’, ‘ALB’) having an insertion in the region of amino acid residues corresponding to said region of His-450 to Asp-462 in human prothrombin. The conserved residues histidine at position 450 and aspartic acid at position 462 are highlighted.

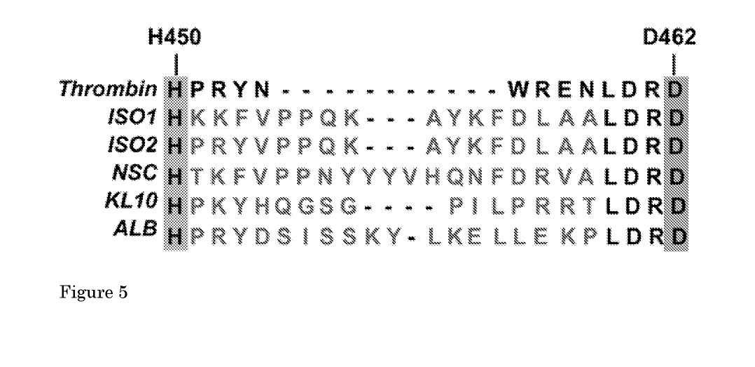

FIG. 6. Inhibition of chromogenic thrombin activity by the direct thrombin inhibitor dabigatran—serie 1. Peptidyl substrate conversion (S-2238; 100 μM) by 5 nM plasma-derived thrombin (‘IIa’; panel A), activated plasma-derived prothrombin (‘pd-IIa’; panel B), recombinant thrombin (‘r-IIa’; panel C), recombinant thrombin variant ISO1 (‘ISO1’; panel D), or recombinant thrombin variant NSC (‘NSC’; panel E) in the presence of increasing concentrations (20 nM-20 μM) dabigatran. IC50 concentrations were obtained by fitting the S-2238 conversion (mOD/min) by nonlinear regression using the Graphpad Prism software suite. All data points represent the average of two independent experiments. Panel F: The substrate conversion (velocity) was plotted as the ratio of incubations in the absence of inhibitor. It is clearly shown in Panel F that the variants show a higher normalized velocity than the control.

FIG. 7. Inhibition of chromogenic thrombin activity by the direct thrombin inhibitor dabigatran—serie 2. Peptidyl substrate conversion (S-2238; 100 μM) by 5 nM plasma-derived thrombin (‘IIa’; panel A), recombinant thrombin (‘r-IIa’; panel B), recombinant thrombin variant ALB (‘ALB’; panel C), recombinant thrombin variant KL10 (‘KL10’; panel D), or recombinant thrombin variant ISO2 (‘ISO2’; panel E) in the presence of increasing concentrations (20 nM-20 μM) dabigatran. IC50 concentrations were obtained by fitting the S-2238 conversion (mOD/min) by nonlinear regression using the Graphpad Prism software suite. All data points represent the average of two independent experiments. Panel F: The substrate conversion (velocity) was plotted as the ratio of incubations in the absence of inhibitor. It is clearly shown in Panel F that the variants show a higher normalized velocity than the control.

FIG. 8. Crystal structure (PDB 1KTS) of thrombin in complex with dabigatran (Hauel et al., J Med Chem 45: 1757-1766 (2002)). The active site residues His406, Asp462, and Ser568 and the dabigatran interaction residues Tyr410, Leu459, Ile542, Asp562, Trp590, and Gly591 are shown in stick figures. The location of the S4 subpocket of the active site is marked by an oval, and residue Ile542 is indicated.

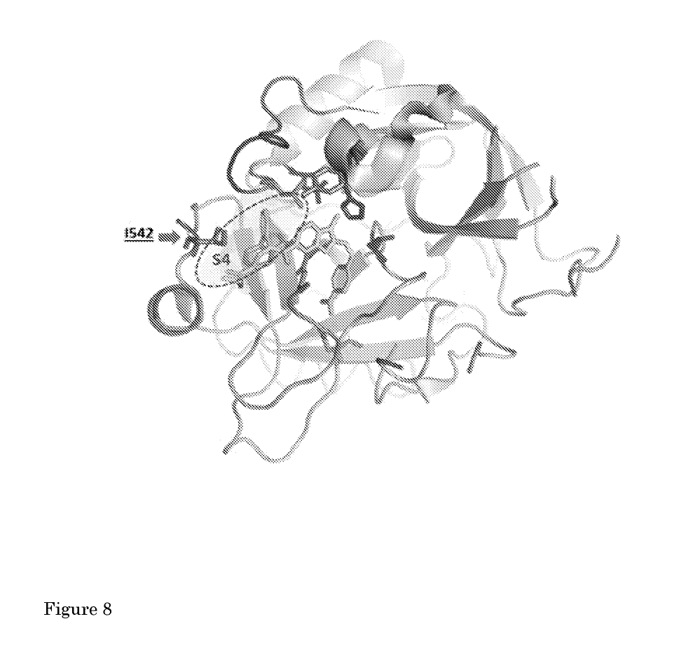

FIG. 9. Inhibition of chromogenic thrombin activity by the direct thrombin inhibitor dabigatran—serie 3. Peptidyl substrate conversion (S-2238; 100 μM) by 5 nM plasma-derived thrombin (panel A), recombinant thrombin (panel B), recombinant thrombin variant I542F (panel C), recombinant thrombin variant I542A (panel D), recombinant thrombin variant I542E (panel E), or recombinant thrombin variant I542S (panel F) in the presence of increasing concentrations (20 nM-20 μM) dabigatran. IC50 concentrations were obtained by fitting the S-2238 conversion (mOD/min) by nonlinear regression using the Graphpad Prism software suite. All data points represent the average of two independent experiments.

FIG. 10. Normalized inhibition of chromogenic thrombin activity by the direct thrombin inhibitor dabigatran. Peptidyl substrate conversion (S-2238; 100 μM) by 5 nM plasma-derived thrombin (Ha), recombinant thrombin (‘r-IIa’), recombinant thrombin variant I542A, recombinant thrombin variant I542E, recombinant thrombin variant I542F, or recombinant thrombin variant I542S in the presence of increasing concentrations (20 nM-20 μM) dabigatran. The substrate conversion (velocity) was plotted as the ratio of incubations in the absence of inhibitor. All data points represent the average of two independent experiments. It is clearly shown that the variants show a higher normalized velocity than the control.

FIG. 11. An alignment of the region of amino acid residues from Cys-536 to Cys-550 in human prothrombin (‘Thrombin’, SEQ ID NO:1), Cys-545 to Cys-560 in human factor XI (‘FXIa’, SEQ ID NO:2), Cys-171 to Cys-185 in human trypsin-1 (‘Trypsin’, SEQ ID NO:3), and Cys-345 to Cys-361 in human urokinase-type plasminogen activator (‘uPA’, SEQ ID NO:4). The residues Ile-542 (SEQ ID NO:1), His-552 (SEQ ID NO:2), Gly-177 (SEQ ID NO:3), and Ser-353 (SEQ ID NO:4) are highlighted.

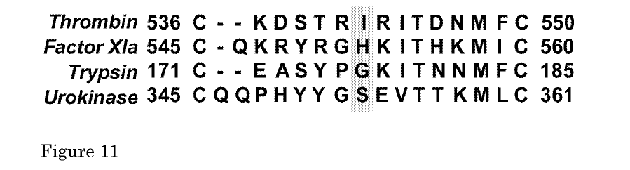

TABLE 1

| Amino | 3- | 1- | Side-chain | Side-chain | Hydropathy | Absorbance | [symbol] at |
|---|---|---|---|---|---|---|---|
| Acid [symbol] | Letter [symbol] | Letter [symbol] | polarity [symbol] | charge (pH 7.4) [symbol] | index [symbol] | λₘₐₓ(nm) [symbol] | λₘₐₓ (×10⁻³ M⁻¹ cm⁻¹) [symbol] |
| Alanine | Ala | A | nonpolar | neutral | 1.8 | | |
| Arginine | Arg | R | Basic polar | positive | −4.5 | | |
| Asparagine | Asn | N | polar | neutral | −3.5 | | |
| Aspartic acid | Asp | D | acidic polar | negative | −3.5 | | |
| Cystine | Cys | C | nonpolar | neutral | 2.5 | 250 | 0.3 |
| Glutamic acid | Glu | E | acidic polar | negative | −3.5 | | |
| Glutamine | Gln | Q | polar | neutral | −3.5 | | |
| Glycine | Gly | G | nonpolar | neutral | −0.4 | | |
| Histidine | His | H | Basic polar | positive(10%) | −3.2 | 211 | 5.9 |
| | | | | neutral(90%) | | | |
| Isoleucine | Ile | I | nonpolar | neutral | 4.5 | | |
| Leucine | Leu | L | nonpolar | neutral | 3.8 | | |
| Lysine | Lys | K | Basic polar | positive | −3.9 | | |
| Methionine | Met | M | nonpolar | neutral | 1.9 | | |
| Phenylalanine | Phe | F | nonpolar | neutral | 2.8 | 257, 206, 188 | 0.2, 9.3, 68.0 |
| Proline | Pro | P | nonpolar | neutral | −1.6 | | |
| Serine | Ser | S | polar | neutral | −0.8 | | |
| Threonine | Thr | T | polar | neutral | −0.7 | | |
| Tryptophan | Trp | W | nonpolar | neutral | −0.9 | 290, 219 | 5.8, 47.0 |
| Tyrosine | Tyr | Y | polar | neutral | −1.3 | 274, 222, 193 | 1.4, 8.0, 48.0 |
| Valine | Val | V | nonpolar | neutral | 4.2 | | |

[symbol]

indicates data missing or illegible when filed

### EXAMPLES

### Example 1: Material and Methods

### Materials:

Direct serine protease inhibitors are obtained from Alsachim and Adooq, and corn trypsin inhibitor is from Haematologic Technologies. FXI-depleted and prothrombin-depleted human plasma, Neoplastin CI plus 10, and TriniCLOT automated APTT are obtained from Diagnostica Stago. The peptidyl substrates S-2238, S-2366, S-2444 and S-2222 are obtained from Chromogenix. All tissue culture reagents are from Life Technologies except insulin-transferrin-sodium selenite (ITS), which is from Roche. Calibrator and fluorescent substrate (FluCa) are from Thrombinoscope BV. Small unilamellar phospholipid vesicles (PCPS) composed of 75% (w/w) hen egg L-phosphatidylcholine and 25% (w/w) porcine brain L-phosphatidylserine (Avanti Polar Lipids) are prepared and characterized as described previously (Higgins et al. 1983. J Biol Chem 258: 6503-6508). General recombinant protein production and purification techniques are as described in Green and Sambrook, Molecular Cloning, 4^{th} edition, July 2012.

### Expression and Purification of Thrombin:

Plasmids (pcDNA3.1(+)) encoding prothrombin variants A (prothrombin (SEQ ID NO:1) with between His-450 and Asp-462 the amino acid sequence of SEQ ID NO:5) and B (prothrombin (SEQ ID NO:1) with between His-450 and Asp-462 the amino acid sequence of SEQ ID NO:6) and wildtype prothrombin are introduced into HEK 293 cells using LipofectAMINE 2000 (Invitrogen) and pSV2neo as the selectable marker plasmid. High expressing clones are selected based on prothrombin-specific ELISA and PT clotting assays, essentially as described (Orcutt et al. 2004. J Biol Chem 279: 54927-54936). Selected clones are expanded into 10-stacked cell factories (Nalge-Nunc, Naperville, Ill.) and cultured in Dulbecco's modified Eagle's medium/F-12 media supplemented with 5 μg/ml ITS and 10 μg/ml Vitamin K. Conditioned media is collected for 5-6 days, centrifuged, and stored at −20° C. in the presence of 1 mM benzamidine.Prothrombin is purified from conditioned media employing Q-Sepharose FF (GE Healthcare), an HQ POROS matrix (Affinity Biologicals), and a ceramic hydroxyapatite matrix (Bio-Rad), essentially as described (Orcutt et al. 2004. J Biol Chem 279: 54927-54936). Purified prothrombin is stored at −20° C. in HBS containing 50% vol/vol glycerol. Protein purity is assessed by SDS-PAGE using pre-cast 4-12% gradient gels (Invitrogen) under reducing conditions followed by staining with Coomassie Brilliant Blue R-250. Thrombin is purified following preparative activation of prothrombin as described (Lundblad et al., 1976. Methods Enzymol 45: 156-176).

### Expression and Purification of FXI:

Plasmids (pcDNA3.1(+)) encoding FXI variants A (FXI (SEQ ID NO:2) with between His-469 and Asp-480 the amino acid sequence of SEQ ID NO:7 and B (FXI (SEQ ID NO:2) with between His-469 and Asp-480 the amino acid sequence of SEQ ID NO:8 and wildtype FXI, fused via their C-terminus to the HPC4-antibody recognition sequence (amino acid sequence EDQVDPRLIDGK) to facilitate purification, are introduced into baby hamster kidney (BHK) cells using LipofectAMINE 2000 (Invitrogen) and pSV2neo as the selectable marker plasmid. High expressing clones are selected based on FXI-specific ELISA and APTT clotting assays, essentially as described for factor V (Toso et al. 2004. J Biol Chem 279: 21643-21650). Selected clones are expanded into triple flasks (Nalge-Nunc, Naperville, Ill.) and cultured in Dulbecco's modified Eagle's medium/F-12 media supplemented with 5 μg/ml ITS and 1.0 mg/ml Albumax (Invitrogen). Conditioned media is collected for 5-6 days, centrifuged, and stored at −20° C. in the presence of 1 mM benzamidine.Conditioned media is thawed at 37° C., pooled, and loaded onto an anti-HPC4 Sepharose column equilibrated in 25 mM Tris, 0.05 M NaCl, 5 mM CaCl2, pH 7.4. The column is washed with the equilibration buffer, and then eluted with 25 mM Tris, 0.5 M NaCl, 5 mM EDTA, pH 7.4, followed by elution with 25 mM Tris, 2 M NaCl, 5 mM EDTA, pH 7.4. Fractions containing FXI activity are pooled, dialyzed versus 20 mM Hepes, 150 mM NaCl, pH 7.4, concentrated by ultrafiltration (Millipore), and the purified protein is stored at −80° C. Protein purity is assessed by SDS-PAGE using pre-cast 4-12% gradient gels (Invitrogen) under reducing conditions followed by staining with Coomassie Brilliant Blue R-250. Factor XIa is purified following preparative activation of FXI as described (Ogawa et al., 2005. J Biol Chem 280: 23523-23530).

Expression and Purification of Urokinase-Type Plasminogen Activator (uPA):

Plasmids (pcDNA3.1(+)) encoding human uPA variants A (uPA (SEQ ID NO:4) with between His-262 and Asp-275 the amino acid sequence of SEQ ID NO:11) and B (uPA (SEQ ID NO:4) with between His-262 and Asp-275 the amino acid sequence of SEQ ID NO:12) and wildtype uPA that are fused via their C-terminus to the HPC4-antibody recognition sequence (amino acid sequence EDQVDPRLIDGK) or to a His-tag (6His: HHHHHH or 12His: HHHHHHHHHHHH) to facilitate purification are introduced into mammalian cells (HEK 293 are BHK) using LipofectAMINE 2000 (Invitrogen) and pSV2neo as the selectable marker plasmid. High expressing clones are selected based on a human uPA-specific ELISA (R&D Systems). Selected clones are expanded into triple flasks (Nalge-Nunc, Naperville, Ill.) and cultured in Dulbecco's modified Eagle's medium/F-12 media supplemented with 5 μg/ml ITS and 1.0 mg/ml Albumax (Invitrogen). Conditioned media is collected for 5-6 days, centrifuged, and stored at −20° C. in the presence of 1 mM benzamidine.

Conditioned media is thawed at 37° C., pooled, and purified using an anti-HPC4 Sepharose essentially as described for the FXI variants. Alternatively, His-tagged uPA variants are purified using employing immobilized metal affinity chromatography. The purified protein is stored at −80° C., and protein purity is assessed by SDS-PAGE using pre-cast 4-12% gradient gels (Invitrogen) under reducing conditions followed by staining with Coomassie Brilliant Blue R-250. uPA variants are activated by human plasmin and purified employing Benzamidine-Sepharose.

### Expression and Purification of Trypsin:

Plasmids (pcDNA3.1(+)) encoding human trypsinogen-1 variants A (trypsin (SEQ ID NO:3) with between His-96 and Asp-107 the amino acid sequence of SEQ ID NO:9) and B (trypsin (SEQ ID NO:3) with between His-96 and Asp-107 the amino acid sequence of SEQ ID NO:10) and wildtype trypsin (SEQ ID NO:3) in which the leader sequence is modified such that it lacks a trypsin-like enzyme cleavage site (see Patent EP 1141263 A1) and that are fused via their C-terminus to the HPC4-antibody recognition sequence (amino acid sequence EDQVDPRLIDGK) or to a His-tag (6His: HHHHHH or 12His: HHHHHHHHHHHH) to facilitate purification are introduced into HEK 293 cells using LipofectAMINE 2000 (Invitrogen) and pSV2neo as the selectable marker plasmid. High expressing clones are selected based on a human trypsinogen-specific ELISA (MyBioSource). Selected clones are expanded into triple flasks (Nalge-Nunc, Naperville, Ill.) and cultured in Dulbecco's modified Eagle's medium/F-12 media supplemented with 5 μg/ml ITS and 1.0 mg/ml Albumax (Invitrogen). Conditioned media is collected for 5-6 days, centrifuged, and stored at −20° C. in the presence of 1 mM benzamidine.

Conditioned media is thawed at 37° C., pooled, and purified using an anti-HPC4 Sepharose essentially as described for the FXI variants. Alternatively, His-tagged trypsinogen variants are purified using employing immobilized metal affinity chromatography. The purified protein is stored at −80° C., and protein purity is assessed by SDS-PAGE using pre-cast 4-12% gradient gels (Invitrogen) under reducing conditions followed by staining with Coomassie Brilliant Blue R-250. Trypsin-1 variants are prepared from enterokinase-dependent cleavage of trypsinogen-1 variants and purified employing SP- or Benzamidine-Sepharose.

### Example 2: Inhibition of Serine Proteases by Direct Serine Protease Inhibitors

Initially, the kinetics of peptidyl substrate hydrolysis (S-2388, S-2366, S-2444 or S-2222 as specific substrates for thrombin, FXIa, urokinase-type plasminogen activator or trypsin, respectively) are measured using increasing concentrations of substrate (10-500 μM) in the presence of the aforementioned thrombin, FXIa, urokinase-type plasminogen activator or trypsin variants and wildtype. The ability of the direct serine protease inhibitors to bind and inhibit the serine proteases is tested by assessing the inhibitory constant (Ki) assuming classical competitive inhibition by initial velocity measurements of peptidyl substrate hydrolysis using increasing concentrations of direct inhibitor (1 nM-10 μM) at fixed concentrations of peptidyl substrate (at or above the Km) and enzyme. All kinetic measurements are performed in 20 mM Hepes, 0.15 M NaCl, 0.1% (w/v) polyethylene glycol 8000, 2 mM CaCl2, pH 7.5.

### Example 3: Thrombin Generation Assays

Thrombin generation is adapted from protocols earlier described (Hemker et al., 2003. Pathophysiol Haemost Thromb 33: 4-15). Briefly, thrombin generation curves are obtained by supplementing prothrombin- or FXI-deficient plasma with corn trypsin inhibitor (70 μg/ml), PCPS (20 μM) and substrate buffer (Fluca). Thrombin formation is initiated by the addition of 1 Unit (specific clotting activity) of a thrombin A or B variant, FXIa A or B variant, and wildtype thrombin or FXIa, which is premixed with the direct serine protease inhibitor. In an alternative set-up, the zymogen forms of the protein variants are assessed. To do so, prothrombin- or FXI-depleted plasma is supplemented with tissue factor (TF (Innovin), 2 or 20 pM final), corn trypsin inhibitor (70 μg/ml), PCPS (20 μM), the direct inhibitor, and 1 Unit (specific clotting activity) of recombinant prothrombin or FXI variant, respectively. Thrombin formation is initiated by the addition of Fluca to the plasma. Thrombin formation is determined every 20s for 30 minutes and corrected for the calibrator, using the Thrombinoscope software (Thrombinoscope BV). The lag time, mean endogenous thrombin potential (the area under the thrombin generation curve), time to peak, and peak thrombin generation are calculated from at least 3 individual experiments.

### Example 4: Clot Lysis Time Assessment for Urokinase-Type Plasminogen Activator Variants

The clot lysis time is essentially assessed as described previously (Mosnier et al., 2001. Thromb Haemost 86: 1035-1039). Briefly, tissue factor (TF, Innovin) and PCPS are incubated at 37° C. for 1 hour in 25 mM Hepes, 137 mM NaCl, 3.5 mM KCl, 0.1% BSA, pH 7.4. The TF/PCPS mixture (0.5 pM/20 μM final) is incubated with plasma (50% v/v), tPA (150 U/ml final), and CTI (70 μg/ml final) for 10 minutes at 37° C. Coagulation is started with Ca2+ (17 mM final) that was pre-incubated at 37° C. The clot formation and the subsequent lysis are monitored by measuring the absorbance at 405 nm for 4 hours at 37° C. in a SpectraMax M2e microplate reader. The clot lysis time is defined as the average of the clear to turbid transition to the average of the turbid to clear transition, which is determined by a sigmoidal fit of the turbidity plots using GraphPad Prism 5.

### Example 5: Recombinant Prothrombin Having an Insertion in Region His450-Asp462 of SEQ ID NO:1

### Materials and Methods

The direct thrombin inhibitor dabigatran was obtained from Alsachim (France). Plasma-derived human prothrombin, plasma-derived human thrombin (alpha-thrombin, IIa), plasma-derived human factor Xa, plasma-derived human factor Va, and the thrombin inhibitor dansylarginine N-(3-ethyl-1,5-pentanediyl)amide (DAPA) were from Haematologic Technologies. Prothrombin-depleted human plasma and the prothrombin time clotting assay reagent STA-Neoplastine CI plus 10 were obtained from Diagnostica Stago. The peptidyl substrate S-2238 was obtained from Chromogenix® (Instrumentation Laboratory). All tissue culture reagents were from Life Technologies (Thermo Fisher Scientific), except insulin-transferrin-sodium selenite (ITS), which is from Roche. General recombinant protein production and purification techniques were as described in Green and Sambrook, Molecular Cloning, 4th edition, July 2012. Small unilamellar phospholipid vesicles (PCPS) composed of 75% (w/w) hen egg L-phosphatidylcholine and 25% (w/w) porcine brain L-phosphatidylserine (Avanti Polar Lipids, Inc., US) were prepared and characterized as described previously (Higgins et al., J Biol Chem 258: 6503-6508 (1983)).

Plasmids (pcDNA3.1(+)) encoding wild-type prothrombin (SEQ ID NO:1) and prothrombin variants with between His-450 and Asp-462 the amino acid sequence of (i) SEQ ID NO:5 (prothrombin ISO1, derived from the homologous region in Pseudonaja textilis isoform factor X), (ii) SEQ ID NO:13 (prothrombin ISO2), (iii) SEQ ID NO:14 (prothrombin NSC, derived from the homologous region in Notechis scutatus venom factor X), (iv) SEQ ID NO:15 (prothrombin KL10, derived from the homologous region in human kallikrein 10), or (v) SEQ ID NO:16 (prothrombin ALB, derived from human albumin) were introduced into HEK 293 cells using LipofectAMINE 2000® (Invitrogen). High expressing clones were selected based on prothrombin-specific ELISA and prothrombin-time clotting assays, essentially as described in Orcutt et al., J Biol Chem, 279: 54927-54936 (2004). Selected clones were expanded into 175 cm² flasks and cultured in Dulbecco's modified Eagle's medium/F-12 media supplemented with 5 μg/ml ITS and 10 μg/ml Vitamin K. Conditioned media was collected for 24 hours, concentrated using a 30 kDa spin-filter into HEPES-buffered saline, pH 7.5, and stored at −20° C. in 50% vol/vol glycerol. The prothrombin antigen concentration was determined using a paired antibody ELISA for the detection of prothrombin (CL20111K, Cedarlane Laboratories). The prothrombin activity was determined employing a prothrombin-specific one-stage prothrombin time clotting assay using the STA-Neoplastin CI plus 10 reagent in prothrombin-deficient plasma, employing known concentrations of prothrombin as standard. The specific activity (U/mg) was derived from the ratio of the prothrombin activity (U/ml) over the prothrombin antigen concentration (mg/ml).

### Inhibition of Thrombin by the Direct Thrombin Inhibitor Dabigatran

Plasma-derived prothrombin (Haematologic Technologies) or recombinant wild-type prothrombin or the recombinant prothrombin variants described in the previous paragraph (ISO1, ISO2, NSC, KL10, and ALB) (125 nM) were activated into thrombin by incubations with prothrombinase (1 nM factor Xa, 50 nM factor Va, 50 μM PCPS, 5 mM calcium) in the presence of 10 μM DAPA during 5 minutes at ambient temperature. Samples were subsequently quenched in EDTA (25 mM final) and diluted to 5 nM (final) in buffer containing EDTA (50 mM), NaCl (150 mM), 0.1% PEG8000 and HEPES (20 mM), pH 7.5. The ability of the direct thrombin inhibitor dabigatran to bind and inhibit activated plasma-derived prothrombin (pd-IIa) or recombinant activated prothrombin (r-IIa) or the recombinant activated prothrombin variants was tested by assessing the half maximal inhibitory concentration (IC50) by initial velocity measurements of the peptidyl substrate S-2238 hydrolysis using increasing concentrations of dabigatran (20 nM-20 μM) at a fixed concentration of S-2238 (100 μM). Residual chromogenic activity towards the peptidyl substrate was determined during 10 minutes in a microplate reader (SpectraMax M2e, Molecular Devices) set at A405 nm. IC50 concentrations were obtained by fitting the S-2238 conversion (mOD/min) by nonlinear regression using the Graphpad Prism 6 software suite. The same experiment was performed using plasma-derived thrombin (IIa, Haematologic Technologies) as a control.

### Results

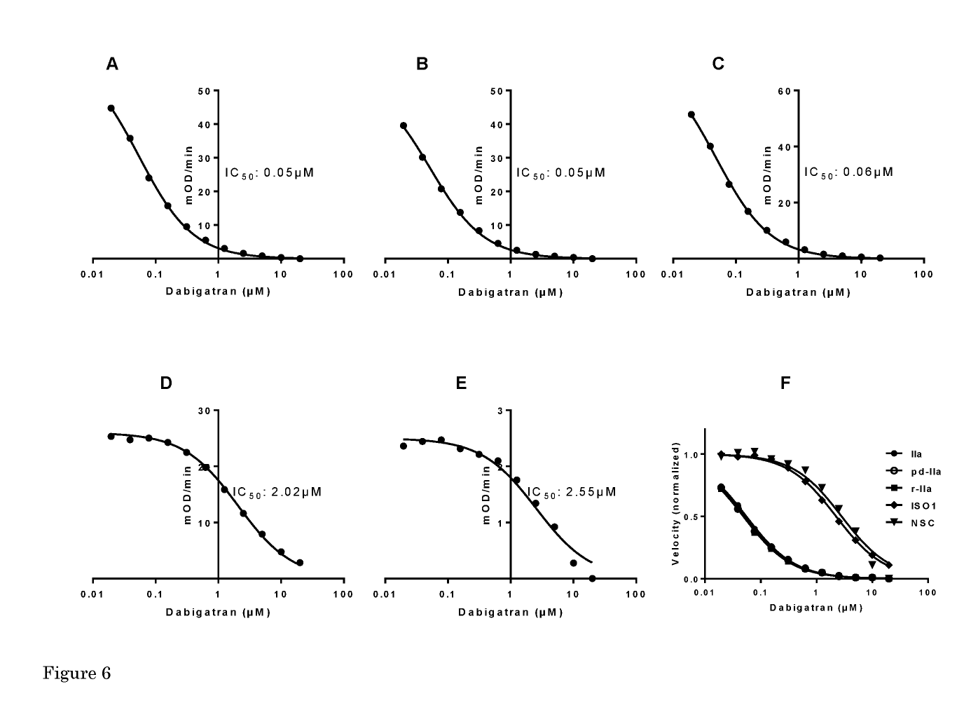

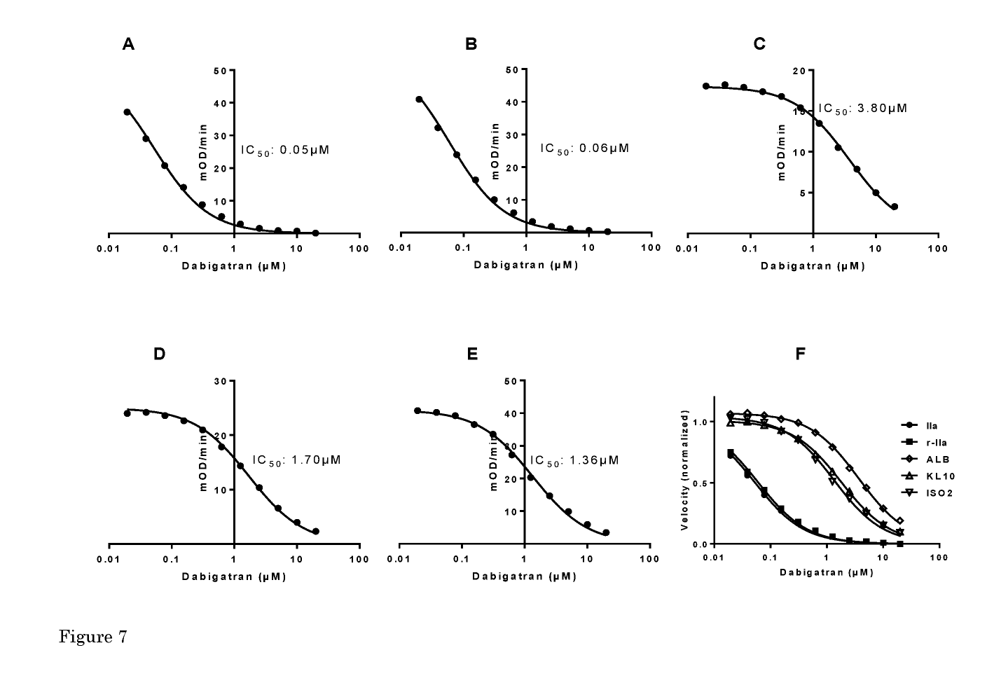

The results of these experiments are displayed in Table 2 and FIGS. 6 and 7. From all this it follows that an insertion in the claimed amino acid residue region of prothrombin provides for desensitization towards direct thrombin inhibitors such as dabigatran, while still having clotting potential or a coagulant effect.

TABLE 2

| | Specific | | Dabigatran |
|---|---|---|---|
| Prothrombin | Activity × | Chromogenic | Inhibition |
| Variant | 10⁻³ U/mg | Activity % | IC₅₀ (μM) |
| IIa | n.d. | 92 | 0.05 |
| r-IIa | 3.88-4.15 | 96 | 0.06 |
| ISO1 | 0.41 | 39 | 2.02 |
| ISO2 | 0.50 | 70 | 1.36 |
| NSC | 0.14 | 4 | 2.55 |
| KL10 | 0.69 | 43 | 1.70 |
| ALB | 1.17 | 30 | 3.80 |

Table 2 shows the characteristics of the different prothrombin variants. The plasma-derived thrombin is indicated as IIa and the recombinant wild-type thrombin as r-IIa. The specific activity (U/mg) is derived from the ratio of the prothrombin activity (U/ml), determined using a prothrombin-specific one-stage prothrombin-time clotting assay, over the prothrombin antigen concentration (mg/ml). The percentage (%) of chromogenic activity denotes the S-2238 conversion of each prothrombin variant related to a standard curve of purified plasma-derived thrombin. The half maximal inhibitory concentration (IC50) displays the concentration of dabigatran required to inhibit 50% of the chromogenic activity of 5 nM thrombin variant.

### Example 6. Recombinant Prothrombin Having an Amino Acid Residue Replacement or Substitution at Position Ile-542 of SEQ ID NO:1

### Materials and Methods

The direct thrombin inhibitor dabigatran was obtained from Alsachim (France). Plasma-derived human prothrombin, plasma-derived human thrombin (alpha-thrombin, IIa), plasma-derived human factor Xa, plasma-derived human factor Va, and the thrombin inhibitor dansylarginine N-(3-ethyl-1,5-pentanediyl)amide (DAPA) were from Haematologic Technologies. Prothrombin-depleted human plasma and the prothrombin time clotting assay reagent STA-Neoplastine CI plus 10 were obtained from Diagnostica Stago. The peptidyl substrate S-2238 was obtained from Chromogenix® (Instrumentation Laboratory). All tissue culture reagents were from Life Technologies (Thermo Fisher Scientific), except insulin-transferrin-sodium selenite (ITS), which was from Roche. General recombinant protein production and purification techniques were as described in Green and Sambrook, Molecular Cloning, 4th edition, July 2012. Small unilamellar phospholipid vesicles (PCPS) composed of 75% (w/w) hen egg L-phosphatidylcholine and 25% (w/w) porcine brain L-phosphatidylserine (Avanti Polar Lipids, Inc., US) were prepared and characterized as described previously (Higgins et al., J Biol Chem 258: 6503-6508 (1983)).

Plasmids (pcDNA3.1(+)) encoding wild-type prothrombin (SEQ ID NO:1) and prothrombin variants in which the Isoleucine at amino acid residue position 542 of SEQ ID NO:1 were replaced by Alanine (I542A, no side-chain), Serine (1542S, small side-chain), Phenylalanine (1542F, large bulky side-chain), or Glutamic acid (I542E, charged side-chain) were introduced into HEK 293 cells using LipofectAMINE 2000® (Invitrogen). High expressing clones were selected based on prothrombin-specific ELISA and prothrombin-time clotting assays, essentially as described in Orcutt et al., J Biol Chem, 279: 54927-54936 (2004). Selected clones were expanded into 175 cm² flasks and cultured in Dulbecco's modified Eagle's medium/F-12 media supplemented with 5 μg/ml ITS and 10 μg/ml Vitamin K. Conditioned media was collected for 24 hours, concentrated using a 30 kDa spin-filter into HEPES-buffered saline, pH 7.5, and stored at −20° C. in 50% vol/vol glycerol. The prothrombin antigen concentration was determined using a paired antibody ELISA for the detection of prothrombin (CL20111K, Cedarlane Laboratories). The prothrombin activity was determined employing a prothrombin-specific one-stage prothrombin time clotting assay using the STA-Neoplastin CI plus 10 reagent in prothrombin-deficient plasma, employing known concentrations of prothrombin as standard. The specific activity (U/mg) was derived from the ratio of the prothrombin activity (U/ml) over the prothrombin antigen concentration (mg/ml).

### Inhibition of Thrombin by the Direct Thrombin Inhibitor Dabigatran

Recombinant wild-type prothrombin or the recombinant prothrombin variants (1542S, I542A, I542F and I542E) (125 nM) were activated into thrombin by incubations with prothrombinase (1 nM factor Xa, 50 nM factor Va, 50 μM PCPS, 5 mM calcium) in the presence of 10 μM DAPA during 5 minutes at ambient temperature. Samples were subsequently quenched in EDTA (25 mM final) and diluted to 5 nM final in buffer containing EDTA (50 mM), NaCl (150 mM), 0.1% PEG8000 and HEPES (20 mM), pH 7.5. The ability of the direct thrombin inhibitor dabigatran to bind and inhibit recombinant activated prothrombin (r-IIa) or the recombinant activated prothrombin variants was tested by assessing the half maximal inhibitory concentration (IC50) by initial velocity measurements of the peptidyl substrate S-2238 hydrolysis using increasing concentrations of direct inhibitor (20 nM-20 μM) at a fixed concentrations of S-2238 (100 μM). Residual chromogenic activity towards the peptidyl substrate was determined during 10 minutes in a microplate reader (SpectraMax M2e, Molecular Devices) set at A405 nm. IC50 concentrations were obtained by fitting the S-2238 conversion (mOD/min) by nonlinear regression using the Graphpad Prism 6 software suite. The same experiment was performed using plasma-derived thrombin (IIa, Haematologic Technologies) as a control.

### Results

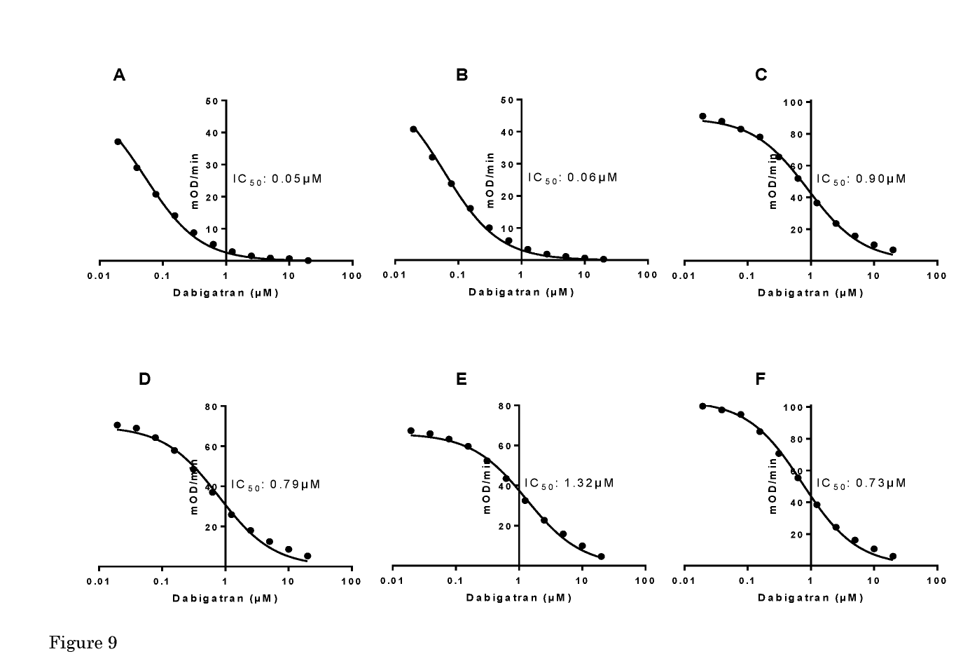

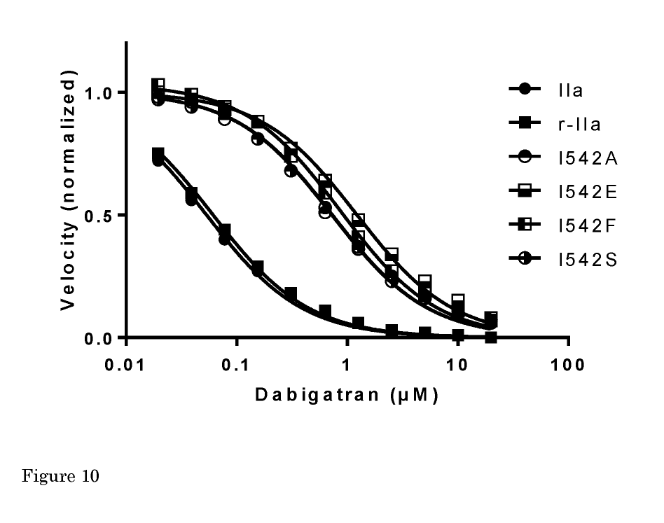

The results of these experiments are displayed in Table 3 and FIGS. 9 and 10. From all this it follows that a replacement, substitution or mutation at amino acid residue position Ile-542 of SEQ ID NO:1 provides for desensitization towards direct thrombin inhibitors such as dabigatran, while still having clotting potential or a coagulant effect.

TABLE 3

| | Specific | | Dabigatran |
|---|---|---|---|
| Prothrombin | Activity × | Chromogenic | Inhibition |
| Variant | 10⁻³ U/mg | Activity % | IC₅₀ (μM) |
| IIa | n.d. | 92 | 0.05 |
| r-IIa | 3.88-4.15 | 96 | 0.06 |
| I542S | 0.30 | 183 | 0.73 |
| I542A | 0.40 | 127 | 0.79 |
| I542F | 0.74 | 156 | 0.90 |
| I542E | 0.24 | 119 | 1.32 |

Table 3 shows the characteristics of the prothrombin variants. The plasma-derived thrombin is indicated as IIa and the recombinant wild-type thrombin as r-IIa. The specific activity (U/mg) is derived from the ratio of the prothrombin activity (U/ml), determined using a prothrombin-specific one-stage prothrombin-time clotting assay, over the prothrombin antigen concentration (mg/ml). The percentage (%) of chromogenic activity denotes the S-2238 conversion of each prothrombin variant related to a standard curve of purified plasma-derived thrombin. The half maximal inhibitory concentration (IC50) displays the concentration of dabigatran required to inhibit 50% of the chromogenic activity of 5 nM thrombin variant.

1. A recombinant protein comprising a serine protease, said serine protease comprising an insertion of an amino acid residue in an outer-surface peptide structure; wherein the serine protease polypeptide is not a coagulation factor X polypeptide or naturally processed or activated forms thereof.

2. The protein according to claim 1, wherein said peptide structure is a region of amino acid residues corresponding to the region of amino acid residues between His-450 and Asp-462 of SEQ ID NO: 1.

3. The protein according to claim 1, wherein the serine protease is selected from the group consisting of thrombin, coagulation factor XIa, trypsin and urokinase-type plasminogen activator; and wherein said peptide structure is:
a region of amino acid residues between Gly-427 and Asp-462, of SEQ ID NO: 1 of thrombin;
a region of amino acid residues between Val-463 and Asp-480 of SEQ ID NO: 2 of coagulation factor XIa;
a region of amino acid residues between Leu-73 and Asp-107 of SEQ ID NO: 3 of trypsin; and
a region of amino acid residues between Val-237 and Asp-275, of SEQ ID NO:4 of urokinase-type plasminogen activator.

4. The protein according to claim 1, wherein the insertion comprises 1-50 amino acid residues.

5. The protein according to claim 1, wherein the insertion comprises between 4 and 50 amino acid residues.

6. The protein according to claim 2, wherein the insertion of an amino acid residue is combined with a replacement of at least 5 amino acid residues in said region.

7. The protein according to claim 2, wherein said region, after insertion and/or replacement, has the amino acid sequence of:
SEQ ID NO: 5, SEQ ID NO:6, SEQ ID NO: 13, SEQ ID NO: 14, SEQ ID NO: 15 or SEQ ID NO: 16 between His-450 and Asp-462 of SEQ ID NO:1;
SEQ ID NO: 7 or SEQ ID NO: 8 between His-469 and Asp-480 of SEQ ID NO:2; SEQ ID NO: 9 or SEQ ID NO: 10 between His-96 and Asp-107 of SEQ ID NO:3; and
and/or SEQ ID NO: 11 or SEQ ID NO: 12 between His-262 and Asp-275 of SEQ ID NO:4.

8. A recombinant protein comprising a serine protease polypeptide having an amino acid residue replacement on an amino acid residue position corresponding to Ile-542 of SEQ ID NO:1, wherein the serine protease polypeptide is not a coagulation factor X polypeptide or naturally processed or activated forms thereof.

9. A nucleic acid molecule comprising a DNA sequence that encodes the protein according to claim 1.

10. An expression vector comprising the nucleic acid molecule according to claim 9.

11. A host cell comprising the nucleic acid molecule according to claim 9.

12. A pharmaceutical composition comprising the protein according to claim 1 and a pharmaceutically acceptable carrier.

13. (canceled)

14. The protein according to claim 1, wherein the protein comprises thrombin or coagulation factor XIa.

15. A method of reversing an anti-coagulant effect of a coagulation inhibitor in a subject, said method comprising administering to said subject a therapeutically effective amount of
the protein according to claim 1, wherein the protein comprises thrombin or coagulation factor XIa.

16. (canceled)

17. The protein according to claim 1, wherein the protein comprises trypsin.

18. A method of reversing the inhibition of peptide bond hydrolysis of a trypsin inhibitor in a subject, said method comprising administering to said subject a therapeutically effective amount of the protein according to claim 1 wherein the protein comprises trypsin.

19. (canceled)

20. (canceled)

21. The protein according to claim 1, wherein the protein comprises urokinase-type plasminogen activator.


SEQUENCE LISTING (40 sequences)

SEQ ID NO: 1 - protein, 622 aa, Homo sapiens
MAHVRGLQLPGCLALAALCSLVHSQHVFLAPQQARSLLQRVRRANTFLEEVRKGNLERECVEETCSYEEAFEALESSTATDVFWAKYTACETARTPRDKLAACLEGNCAEGLGTNYRGHVNITRSGIECQLWRSRYPHKPEINSTTHPGADLQENFCRNPDSSTTGPWCYTTDPTVRRQECSIPVCGQDQVTVAMTPRSEGSSVNLSPPLEQCVPDRGQQYQGRLAVTTHGLPCLAWASAQAKALSKHQDFNSAVQLVENFCRNPDGDEEGVWCYVAGKPGDFGYCDLNYCEEAVEEETGDGLDEDSDRAIEGRTATSEYQTFFNPRTFGSGEADCGLRPLFEKKSLEDKTERELLESYIDGRIVEGSDAEIGMSPWQVMLFRKSPQELLCGASLISDRWVLTAAHCLLYPPWDKNFTENDLLVRIGKHSRTRYERNIEKISMLEKIYIHPRYNWRENLDRDIALMKLKKPVAFSDYIHPVCLPDRETAASLLQAGYKGRVTGWGNLKETWTANVGKGQPSVLQVVNLPIVERPVCKDSTRIRITDNMFCAGYKPDEGKRGDACEGDSGGPFVMKSPFNNRWYQMGIVSWGEGCDRDGKYGFYTHVFRLKKWIQKVIDQFGE

SEQ ID NO: 2 - protein, 625 aa, Homo sapiens
MIFLYQVVHFILFTSVSGECVTQLLKDTCFEGGDITTVFTPSAKYCQVVCTYHPRCLLFTFTAESPSEDPTRWFTCVLKDSVTETLPRVNRTAAISGYSFKQCSHQISACNKDIYVDLDMKGINYNSSVAKSAQECQERCTDDVHCHFFTYATRQFPSLEHRNICLLKHTQTGTPTRITKLDKVVSGFSLKSCALSNLACIRDIFPNTVFADSNIDSVMAPDAFVCGRICTHHPGCLFFTFFSQEWPKESQRNLCLLKTSESGLPSTRIKKSKALSGFSLQSCRHSIPVFCHSSFYHDTDFLGEELDIVAAKSHEACQKLCTNAVRCQFFTYTPAQASCNEGKGKCYLKLSSNGSPTKILHGRGGISGYTLRLCKMDNECTTKIKPRIVGGTASVRGEWPWQVTLHTTSPTQRHLCGGSIIGNQWILTAAHCFYGVESPKILRVYSGILNQSEIKEDTSFFGVQEIIIHDQYKMAESGYDIALLKLETTVNYTDSQRPICLPSKGDRNVIYTDCWVTGWGYRKLRDKIQNTLQKAKIPLVTNEECQKRYRGHKITHKMICAGYREGGKDACKGDSGGPLSCKHNEVWHLVGITSWGEGCAQRERPGVYTNVVEYVDWILEKTQAV

SEQ ID NO: 3 - protein, 247 aa, Homo sapiens
MNPLLILTFVAAALAAPFDDDDKIVGGYNCEENSVPYQVSLNSGYHFCGGSLINEQWVVSAGHCYKSRIQVRLGEHNIEVLEGNEQFINAAKIIRHPQYDRKTLNNDIMLIKLSSRAVINARVSTISLPTAPPATGTKCLISGWGNTASSGADYPDELQCLDAPVLSQAKCEASYPGKITSNMFCVGFLEGGKDSCQGDSGGPVVCNGQLQGVVSWGDGCAQKNKPGVYTKVYNYVKWIKNTIAANS

SEQ ID NO: 4 - protein, 431 aa, Homo sapiens
MRALLARLLLCVLVVSDSKGSNELHQVPSNCDCLNGGTCVSNKYFSNIHWCNCPKKFGGQHCEIDKSKTCYEGNGHFYRGKASTDTMGRPCLPWNSATVLQQTYHAHRSDALQLGLGKHNYCRNPDNRRRPWCYVQVGLKPLVQECMVHDCADGKKPSSPPEELKFQCGQKTLRPRFKIIGGEFTTIENQPWFAAIYRRHRGGSVTYVCGGSLISPCWVISATHCFIDYPKKEDYIVYLGRSRLNSNTQGEMKFEVENLILHKDYSADTLAHHNDIALLKIRSKEGRCAQPSRTIQTICLPSMYNDPQFGTSCEITGFGKENSTDYLYPEQLKMTVVKLISHRECQQPHYYGSEVTTKMLCAADPQWKTDSCQGDSGGPLVCSLQGRMTLTGIVSWGRGCALKDKPGVYTRVSHFLPWIRSHTKEENGLAL

SEQ ID NO: 5 - protein, 19 aa, Artificial Sequence
  de-inhibitory insert
KKFVPPQKAYKFDLAALDR

SEQ ID NO: 6 - protein, 15 aa, Artificial Sequence
  de-inhibitory insert
PPQKAYKFDLAALDR

SEQ ID NO: 7 - protein, 19 aa, Artificial Sequence
  de-inhibitory insert
KKFVPPQKAYKFDLAASGY

SEQ ID NO: 8 - protein, 15 aa, Artificial Sequence
  de-inhibitory insert
PPQKAYKFDLAASGY

SEQ ID NO: 9 - protein, 19 aa, Artificial Sequence
  de-inhibitory insert
KKFVPPQKAYKFDLAALNN

SEQ ID NO: 10 - protein, 21 aa, Artificial Sequence
  de-inhibitory insert
KKFVPPSQEFYEKFDLVSLNN

SEQ ID NO: 11 - protein, 19 aa, Artificial Sequence
  de-inhibitory insert
KKFVPPQKAYKFDLAAHHN

SEQ ID NO: 12 - protein, 15 aa, Artificial Sequence
  de-inhibitory insert
PPQKAYKFDLAAHHN

SEQ ID NO: 13 - protein, 19 aa, Artificial Sequence
  de-inhibitory insert
PRYVPPQKAYKFDLAALDR

SEQ ID NO: 14 - protein, 22 aa, Artificial Sequence
  de-inhibitory insert
TKFVPPNYYYVHQNFDRVALDR

SEQ ID NO: 15 - protein, 18 aa, Artificial Sequence
  de-inhibitory insert
PKYHQGSGPILPRRTLDR

SEQ ID NO: 16 - protein, 21 aa, Artificial Sequence
  de-inhibitory insert
PRYDSISSKYLKELLEKPLDR

SEQ ID NO: 17 - protein, 12 aa, Artificial Sequence
  HPC4-antibody recognition sequence
EDQVDPRLIDGK

SEQ ID NO: 18 - protein, 6 aa, Artificial Sequence
  His-tag
HHHHHH

SEQ ID NO: 19 - protein, 12 aa, Artificial Sequence
  His-tag
HHHHHHHHHHHH

SEQ ID NO: 20 - protein, 13 aa, Homo sapiens
HPRYNWRENLDRD

SEQ ID NO: 21 - protein, 12 aa, Homo sapiens
HDQYKMAESGYD

SEQ ID NO: 22 - protein, 12 aa, Homo sapiens
HPQYDRKTLNND

SEQ ID NO: 23 - protein, 14 aa, Homo sapiens
HKDYSADTLAHHND

SEQ ID NO: 24 - protein, 21 aa, Artificial Sequence
  de-inhibitory protein segment
HKKFVPPQKAYKFDLAALDRD

SEQ ID NO: 25 - protein, 17 aa, Artificial Sequence
  de-inhibitory protein segment
HPPQKAYKFDLAALDRD

SEQ ID NO: 26 - protein, 21 aa, Artificial Sequence
  de-inhibitory protein segment
HKKFVPPQKAYKFDLAASGYD

SEQ ID NO: 27 - protein, 17 aa, Artificial Sequence
  de-inhibitory protein segment
HPPQKAYKFDLAASGYD

SEQ ID NO: 28 - protein, 21 aa, Artificial Sequence
  de-inhibitory protein segment
HKKFVPPQKAYKFDLAALNND

SEQ ID NO: 29 - protein, 23 aa, Artificial Sequence
  de-inhibitory protein segment
HKKFVPPQSKEYEKFDLVSLNND

SEQ ID NO: 30 - protein, 21 aa, Artificial Sequence
  de-inhibitory protein segment
HKKFVPPQKAYKFDLAAHHND

SEQ ID NO: 31 - protein, 17 aa, Artificial Sequence
  de-inhibitory protein segment
HPPQKAYKFDLAAHHND

SEQ ID NO: 32 - protein, 21 aa, Artificial Sequence
  ISO1 variant
HKKFVPPQKAYKFDLAALDRD

SEQ ID NO: 33 - protein, 21 aa, Artificial Sequence
  ISO2 varainat
HPRYVPPQKAYKFDLAALDRD

SEQ ID NO: 34 - protein, 24 aa, Artificial Sequence
  NSC variant
HTKFVPPNYYYVHQNFDRVALDRD

SEQ ID NO: 35 - protein, 20 aa, Artificial Sequence
  KL10 variant
HPKYHQGSGPILPRRTLDRD

SEQ ID NO: 36 - protein, 23 aa, Artificial Sequence
  ALB variant
HPRYDSISSKYLKELLEKPLDRD

SEQ ID NO: 37 - protein, 15 aa, Homo sapiens
CKDSTRIRITDNMFC

SEQ ID NO: 38 - protein, 16 aa, Homo sapiens
CQKRYRGHKITHKMIC

SEQ ID NO: 39 - protein, 15 aa, Homo sapiens
CEASYPGKITNNMFC

SEQ ID NO: 40 - protein, 17 aa, Homo sapiens
CQQPHYYGSEVTTKMLC